(19) United States
(12) Patent Application Publication
(10) Pub. No.: US 2009/0023794 A1
(43) Pub. Date: Jan. 22, 2009

(54) Use of Sumoylation Inhibitors for the Treatment of Neurodegenerative Disease

(75) Inventors: Jin Xu, Wellesley, MA (US); Nan Zhong, Stony Brook, NY (US)

Correspondence Address: EDWARDS ANGELL PALMER & DODGE LLP, P.O. BOX 55874, BOSTON, MA, 02205, US

(73) Assignee: Caritas St. Elizabeth Medical Center of Boston, Inc., Boston, MA (US)

(21) Appl. No.: 12/090,744

(22) PCT Filed: Oct. 20, 2006
(86) PCT No.: PCT/US06/41084

§ 371 Date: Oct. 8, 2008

Related U.S. Application Data
(60) Provisional application No. 60/729,308, filed on Oct. 21, 2005.

(51) Int. Cl. A61K 31/417; C12Q 1/02; C12Q 1/34; A61P 25/00; G01N 33/53; A61K 31/19

(52) U.S. Cl. 514/400; 435/29; 435/18; 435/7.8; 514/557

(57) ABSTRACT

The invention generally provides screening methods for the identification of therapeutic compounds useful for the treatment of a neurodegenerative disease, and related prophylactic and therapeutic compositions and methods.

### CROSS-REFERENCE TO RELATED APPLICATION

This application claims the benefit of U.S. Provisional Application No. 60/729,308, the entire disclosure of which is hereby incorporated in its entirety.

### BACKGROUND OF THE INVENTION

Parkinson's disease is a common neurodegenerative disorder, second in prevalence only to Alzheimer disease. Parkinson's disease is a heterogeneous disease, and the majority of the cases of Parkinson's disease appear to have sporadic origins. Genetic analyses have identified a number of genes that contribute to Parkinson's disease susceptibility, either in an autosomal dominant or an autosomal recessive pattern. Mutations in PARK1 (alpha-synuclein), PARK2 (parkin), and PARK7 (DJ-1) genes have been shown to cause Parkinson's disease. Regardless of the underlying genetic causation, the symptoms of Parkinson's disease generally include slowed movement (bradykinesia), resting tremor, muscular rigidity, and postural instability. These clinical symptoms result from the near-total destruction of the nigrostriatal dopamine system, which regulates movement. Symptoms of the disease are typically controlled with medications that increase levels of brain dopamine, but these medications have a number of severe side effects. No cure is presently available for Parkinson's disease, and the disorder inevitably progresses to total disability, often accompanied by the general deterioration of all brain functions, and death. Given the inadequacy of current therapies, new methods for treating Parkinson's disease are urgently required.

### SUMMARY OF THE INVENTION

The invention generally provides screening methods for the identification of compositions for the treatment of neurodegenerative diseases (e.g., Parkinson's disease), and related therapeutic and prophylactic compositions and methods.

In one aspect, the invention generally features a method for identifying a compound useful for the treatment of a neurodegenerative disease. The method involves contacting a cell with a candidate compound; and identifying a decrease in sumoylation of a sumo substrate in the cell, where a compound that decreases sumoylation relative to a reference is a compound that treats a neurodegenerative disease. In one embodiment, the protein is selected from the group consisting of pyrimidine tract-binding protein associated factor (PSF), huntingtin, androgen receptor and amyloid precursor protein. In another embodiment, the neurodegenerative disease is selected from the group consisting of Parkinson's disease, Huntington's disease, Alzheimer's disease, Kennedy's Disease, and spinocerebellar ataxia.

In a related aspect, the invention features a method for identifying a compound that increases tyrosine hydroxylase expression. The method involves contacting a cell with a compound; and identifying a decrease in sumoylated substrate in the cell, where a compound that decreases the amount of sumoylated substrate relative to a reference is a compound that increases tyrosine hydroxylase expression. In one embodiment, the sumoylated substrate is PSF. In another embodiment, the cell is a PSF-expressing cell.

In another aspect, the invention features a method for reducing apoptosis. The method involves contacting a cell with a compound; and identifying a decrease in a sumoylated substrate in the cell, where a compound that decreases the amount of sumoylated substrate relative to a reference is a compound that reduces apoptosis. In one embodiment, the sumoylated substrate is PSF. In another embodiment, the cell is a PSF-expressing cell.

In yet another aspect, the invention features a method for identifying a compound useful for the treatment of a neurodegenerative disease. The method involves contacting a PSF-expressing cell with a compound; and identifying a decrease in sumoylated PSF in the cell, where a compound that decreases the amount of sumoylated PSF relative to a reference is a compound that treats a neurodegenerative disease.

In yet another aspect, the invention features a method for identifying a compound that increases tyrosine hydroxylase expression or decreases apoptosis. The method involves contacting a sumoylation substrate (e.g., at least a fragment of a PSF polypeptide) comprising a sumoylation consensus sequence with a compound under conditions that permit binding; and detecting binding of the compound to the consensus sequence, where a compound that specifically binds the substrate increases tyrosine hydroxylase expression or decreases apoptosis. In one embodiment, the polypeptide comprises at least the following amino acid sequence: _{ψ}KXD, where _{ψ} is a hydrophobic amino acid, K is lysine, X is any amino acid, and D is a glutamic acid. In another embodiment, the fragment is fixed to a solid substrate. In another embodiment, the method is carried out in vitro. In another embodiment, the method is carried out in a cell in vivo. In yet another embodiment, the tyrosine hydroxylase expression increases by at least 5%, 10%, 25%, 50%, 75%, or 100%. In still another embodiment, the cell is at risk of apoptosis. In yet another embodiment, the compound reduces apoptosis in a cell at risk thereof. In yet another embodiment, apoptosis is reduced by at least 5%, 10%, 25%, 50%, 75%, or 100%.

In another aspect, the invention features a method of identifying a compound that modulates sumoylation. The method involves contacting a cell expressing a sumoylation-responsive promoter operably linked to a detectable reporter with a candidate compound; and detecting an alteration in the expression level of the detectable reporter, thereby identifying the compound as modulating sumoylation. In one embodiment, a polypeptide comprising a sumoylation consensus site binds to the sumoylation-responsive promoter. In another embodiment, the polypeptide when sumoylated increases or decreases expression of the detectable reporter. In another embodiment, the polypeptide when unsumoylated or when having a reduced level of sumoylation increases or decreases expression of the detectable reporter (e.g., horseradish peroxidase, alkaline phosphatase, luciferase, and GFP). In still other embodiments, the promoter is the tyrosine hydroxylase promoter. In still other embodiments, expression of the reporter construct is repressed in the presence of sumoylated PSF.

In yet another aspect, the invention provides a method of identifying a compound that treats Parkinson's disease. The method involves contacting a cell expressing a tyrosine hydroxylase promoter operably linked to a detectable reporter with a candidate compound; and detecting an alteration in the expression level of the detectable reporter, thereby identifying the compound as modulating sumoylation.

In another aspect, the invention features a method for identifying a compound that reduces PSF sumoylation. The method involves contacting a PSF polypeptide comprising a sumoylation consensus sequence with a compound under conditions that permit SUMO ligation; and detecting a decrease in PSF sumoylation in the presence of the compound relative to a control condition. In one embodiment, the PSF polypeptide of step (a) is further contacted with a SUMO activating enzyme, a SUMO conjugating enzyme, and a SUMO-protein ligase. In another embodiment, the compound is a peptide comprising a sumoylation consensus sequence.

In another aspect, the invention features a method for identifying a compound that increases cleavage of SUMO from a sumoylated substrate (e.g., PSF polypeptide). The method involves contacting at least a fragment of a sumoylated substrate with a compound under conditions that permit cleavage of a SUMO moiety; and detecting a decrease in the level of PSF sumoylation in the presence of the compound relative to a reference. In one embodiment, the sumoylated polypeptide comprises at least 10, 95, 35, or 50 amino acids of a PSF polypeptide. In yet another embodiment, PSF sumoylation is reduced by at least 5%, 10%, 25%, 50%, 75%, or 100%.

In another aspect, the invention features a method for treating a subject having a neurodegenerative disease. The method involves administering to the subject a compound that decreases sumoylation.

In another aspect, the invention features a method for enhancing dopamine synthesis in a subject. The method involves administering to the subject an effective amount of a compound that decreases sumoylation of a substrate (e.g., PSF) thereby enhancing dopamine synthesis. In one embodiment, the method enhances dopamine synthesis by at least 5%, 10%, 25%, 50%, 75%, or 100% in the subject. In another embodiment, the compound is identified according to the method of any previous aspect. In yet another embodiment, a decrease in sumoylation is identified in an immunoassay, such as an ELISA. In another embodiment, the sumoylated protein or the Sumo group is linked to a detectable reporter.

In another aspect, the invention features a method for ameliorating Parkinson's disease in a subject. The method involves administering to the subject an effective amount of a compound that decreases PSF sumoylation. In one embodiment, the method reduces neuronal apoptosis by at least 5%, 10%, 25%, 50%, 75%, or 100% in the subject. In another embodiment, the method enhances dopamine synthesis by at least 5%, 10%, 25%, 50%, 75%, or 100% in the subject.

In yet another aspect, the invention provides a method of identifying a compound that treats Parkinson's disease. The method involves contacting a cell expressing a tyrosine hydroxylase promoter operably linked to a detectable reporter with a candidate compound; and detecting an alteration in the expression level of the detectable reporter, thereby identifying the compound as modulating sumoylation.

In yet another aspect, the invention features a method for preventing or ameliorating Parkinson's Disease in a subject (e.g., a human patient). The method involves administering to the subject an effective amount of a histone deacetylase inhibitor that increases tyrosine hydroxylase expression. In one embodiment, the tyrosine hydroxylase inhibitor is selected from the group consisting of trichostatin A, sodium butyrate, and suberoylanilide hydroxamic acid (SAHA).

In another aspect, the invention features a kit for the treatment or prevention of a neurodegenerative disease, the kit comprising a compound that decreases sumoylation.

In yet another aspect, the invention features a kit for the treatment or prevention of a neurodegenerative disease, the kit comprising a compound that increases tyrosine hydroxylase expression.

In yet another aspect, the invention features a pharmaceutical composition containing an effective amount of a compound that increases tyrosine hydroxylase expression that is any one or more of sodium butyrate, trichostatin A, and SAHA.

In yet another aspect, the invention features a packaged pharmaceutical containing an effective amount of an agent that reduces sumoylation of a sumoylation substrate; and instructions for using the agent to treat a neurodegenerative disease.

In various embodiments of any of the above aspects, the cell is a mammalian cell (e.g., a murine or human cell), such as a neuronal cell (e.g., a dopaminergic neuron). In other embodiments of the above aspects, the method decreases neuronal apoptosis. In still other embodiments of the above aspects, the method relieves transcriptional repression. In various embodiments of any of the above aspects, the decrease in sumoylated PSF is identified in an immunoassay, such as an ELISA. In other embodiments of any of the above aspects, the neurodegenerative disease is selected from the group consisting of Parkinson's disease, Huntington's disease, Kennedy's Disease, and spinocerebellar ataxia. In still other embodiments of the above aspects, the method reduces protein sumoylation by at least 5%, 10%, 25%, 50%, 75%, or 100%. In various embodiments of any of the above aspects, the compound is a histone deacetylase inhibitor (e.g., sodium butyrate, trichostatin A, and SAHA).

### BRIEF DESCRIPTION OF THE DRAWINGS

FIGS. 1A-1F show that DJ-1 and PSF transcriptionally regulate human tyrosine hydroxylase, FIG. 1A is a Western blot showing the expression of tyrosine hydroxylase, DJ-1 and β-actin at various time points in CHP-212 cells transfected with a DJ-1 RNAi construct or with a control construct, FIG. 1B is a graph showing the relative tyrosine hydroxylase mRNA levels determined by quantitative real-time PCR (RT-PCR) in CHP-212 and SH-SY5Y cells forty-eight hours after the transfection of control (CTR) or DJ-1 RNAi (DJ-1) constructs. DJ-1 inactivation (>60%) by DJ-1 RNAi was confirmed by western blotting and RT-PCR. The tyrosine hydroxylase mRNA levels from each sample were normalized to β-actin mRNA. Values represent the mean±s.e.m. N=3 experiments *: P=0.007, **: P=0.002 relative to the control by unpaired t test. FIG. 1C is a graph showing L-Dopa production determined by the HPLC analysis in CHP-212 cells transfected with control or DJ-1-specific siRNA. Values are the mean±s.e.m; femtamole of L-Dopa/μg of protein lysate; n=12 per condition. *: P<0.01 relative to the control by one way ANOVA. FIG. 1D (left panel) is a graph showing the relative tyrosine hydroxylase mRNA levels in SH-SY5Y cells stably expressing a vector control (CTR) or the human myc-his tagged wild-type DJ-1 (DJ-1). Values are the mean±s.e.m. N=3; *: P=0.013 relative to the control by unpaired t test. FIG. 1D is a graph showing the relative tyrosine hydroxylase mRNA levels in SH-SY5Y cells stably expressing a vector control (CTR) or the human myc-his tagged wild-type DJ-1 (DJ-1). Values are the mean±s.e.m. n=3 experiments; *: p<0.05 relative to the control by unpaired t test. The side panels are Western blots indicating the expression levels of DJ-1 and β-actin in stable cells. FIG. 1E is a graph showing relative tyrosine hydroxylase mRNA levels in CHP-212 cells at 48 hours after transient transfection of a vector control (CTR) or the human wild-type PSF. Values represent the mean±s.e.m., n=2. FIG. 1F displays the results of ChIP assays showing the binding of the endogenous PSF (left panels) and DJ-1 (right panels) to the human tyrosine hydroxylase promoter in CHP-212 cells, and in the human substantia nigra pars compacta (human SN) tissue. CTR: no input DNA; Input: 0.5% of the total DNA before IP; IgG: species-matched pre-immune control antibodies for IP; PSF or DJ-1: antibodies specifically recognizing PSF or DJ-1. The results were confirmed using 3 different pairs of primers specifically amplifying the human tyrosine hydroxylase promoter sequences. Primers specific for the human GAPDH promoter were used in negative control experiments.

FIGS. 2A-2G show that wild-type DJ-1 inhibits the sumoylation of PSF. FIG. 2A shows an amino acid sequence alignment of a putative sumoylation site in PSF with the consensus site. ψ is typically a hydrophobic residue, and x can be any amino acid. The PSF site is located between residues 337-340. The confirmed sumoylation sites from 2 well-characterized proteins RanGAP1 and HDAC4 (Watts, 2003) were listed for comparison. FIG. 2B shows two Western blots of total proteins modified by endogenous SUMO-2 or 3 (left), or SUMO-1 (right) in stable SH-SY5Y cells expressing an empty vector (CTR) or wild-type DJ-1 (DJ-1). Identical samples were separated in duplicate gels and probed with a rabbit polyclonal anti-SUMO-2/3 or a mouse monoclonal anti-SUMO-1. The membranes were reprobed for β-actin and DJ-1 to confirm equal loading and the overexpression of DJ-1. Note the reduced abundance of high molecular weight SUMO-1-conjugated proteins in cells over-expressing DJ-1. The membranes were re-probed for 13-actin and DJ-1 to confirm equal loading and the overexpression of DJ-1. FIG. 2C is a Western blot showing the amount of SUMO-1-conjugated proteins in the lymphoblast cells from a normal control individual (WT) and Parkinson's disease patients carrying the exon 1-5 deletion (DEL) or the L166P mutation in the DJ-1 gene. The loss of DJ-1 expression in the Parkinson's disease patients was confirmed by reprobing the membrane for DJ-1. The abundance of high molecular weight proteins (>100 kD) from each sample was quantified with the NIH Image J software, and was normalized to that of the WT. (WT=1; DEL=1.47±0.09; L166P=3.07±0.61; p<0.05 by one-way ANOVA, n=3 experiments). FIG. 2D shows Western blots of SUMOylated and total PSF. Total endogenous PSF was immunoprecipitated using 1 mg of denaturing lysates from the SH-SY5Y cells stably expressing equivalent amounts (confirmed in right panels, exo: exogenous myc-his tagged DJ-1; endo: endogenous DJ-1) of indicated myc-his tagged DJ-1 or a vector control (CTR), and then immunoblotted with an anti-SUMO-1 (top left panels) or anti-PSF antibody (bottom left panels). Mouse pre-immune IgG was used as control antibodies for IP. FIG. 2E is a graph showing quantitative analysis of SUMOylated PSF in SH-SY5Y cells stably expressing the wild-type or mutant DJ-1. The levels of SUMO-1-conjugated PSF were normalized to those of total PSF and are represented as ratios to the control (CTR). n=5 experiments for CTR and WT. *:p<0.001 relative to control by one-way ANOVA with post-hoc test. n=3 for samples from D149A, M261 and R98Q. FIG. 2F shows two Western blots of SUMOylated and total PSF in lymphoblast cells from a control (WT) and PD patient carrying the exons 1-5 deletion mutation of DJ-1 (DEL). PSF was immunoprecipitated from 300 μg of cell lysate. The L166P sample was not analyzed due to insufficient materials for immunoprecipitation assays. FIG. 2G is a graph showing a quantitative analysis of the relative SUMO-1-modified PSF in the lymphoblast cells from the patients. Values represent normalized mean±s.e.m, *:p<0.01 by unpaired t test; n=4.

FIGS. 3A and 3B are immunoblots showing that mutations at the SUMO modification site abolish the SUMOylation of PSF. FIG. 3A shows an immunoprecipitation with anti-PSF antibody (IP:PSF) using lysates from SH-SY5Y transfected with equal amount of a control vector, WT, K338A or 1337A PSF, followed by a western blot analysis of SUMO-1-modified PSF species with an anti-SUMO antibody (WB:SUMO-1), an anti-Flag-tagged antibody (WB:Flag) and total immuno-precipitated PSF (PSF). Note that while equivalent amounts of immuno-precipitated transfected Flag-tagged PSF (lower panel) were present, a decrease in SUMO-1 modified PSF species was observed (top panel). FIG. 3B shows two Western blots of total lysate probed with antibodies that recognize the Flag tag (WB:Flag) or that recognize actin (WB:Actin). These results confirm that similar levels of transfected Flag-tagged PSF species were present in the lysates.

FIGS. 4A-4D show that SUMO-1 modification of PSF is required for its repression of the tyrosine hydroxylase promoter. FIG. 4A is a series of nine micrographs showing that there is a decrease in the co-localization of SUMO-1 and SUMO-1-deficient mutant PSF (1337A, K338A) relative to wild-type (WT) PSF. 1 μg of Flag-tagged WT, 1337A, or K338A PSF was co-transfected with 1 μg of HA-tagged SUMO-1 in native SH-SY5Y cells. The cells were fixed twenty-four hours after transfection and double-labeled with a rabbit polyclonal anti-Flag (left) and a mouse monoclonal anti-HA (middle) followed by appropriate fluorescence-conjugated secondary antibodies. Note the reduced merged signal in cells expressing mutant PSF. FIG. 4B is a series of three micrographs showing SH-SY5Y cells expressing WT or mutant PSF visualized by confocal microscopy. Cells stably expressing WT DJ-1 were transfected with 2 μg of Flag-tagged PSF constructs and labeled with a rabbit polyclonal anti-Flag antibody. Of note are the diffused nucleoplasma localization of WT PSF (arrows) and the increased nuclear membrane localization of the PSF mutants. FIG. 4C is a graph showing a quantitative analysis of PSF-transfected cells exhibiting nuclear membrane localized PSF. SH-SY5Y cells stably expressing a control vector (CTR) or myc-his-tagged DJ-1 (DJ-1) were transiently transfected with 2 μg of a vector expressing flag-tagged WT PSF or expressing the indicated PSF mutants. The cells were labeled with an anti-Flag antibody twenty-four hours after the transfection, and the percentage of transfected cells exhibiting nuclear membrane localization of PSF in each condition was scored. N=3 experiments, with more than 200 cells scored per condition per experiment. Values are the means±s.e.m. *: P=0.0003; **: P<0.001 relative to WT in CTR cells; ***: P<0.01 relative to WT in DJ-1 overexpressing cells by one-way ANOVA with post-hoc Student-Neumann-Kiels test. FIG. 4D is a graph showing the relative tyrosine hydroxylase mRNA levels in CHP-212 cells forty-eight hours after transient transfection of a vector control (CTR) or the indicated PSF construct. Values represent the mean±s.e.m., n=2. An equivalent amount of the transfected WT and mutant PSF was confirmed by quantitative PCR.

FIGS. 5A-5C show that DJ-1 prevents the SUMOylation-dependent recruitment of HDAC I by PSF. FIG. 5A is a Western blot showing that mutations that abolish the SUMOylation of PSF disrupt the recruitment of HDAC1. HEK293 cells plated in 10 cm dishes were co-transfected with vector, Flag-tagged WT or mutant PSF (20 μg), and Flag-tagged HDAC1 (20 μg). After lysis, total HDAC1 was immunoprecipitated and the amount of HDAC1-associated transfected PSF was determined by western blotting using an anti-Flag antibody (left panel). Note the equivalent amount of immunoprecipitated HDAC1 from each sample (left panel). Equivalent expression of transfected PSF and HDAC1 was confirmed by Western blot (anti-Flag) of the total lysates (right panel). WT: wild-type PSF; 337: I337A PSF; 338: I338A PSF. FIG. 5B provides the results of ChIP assays showing that HDAC1 was recruited to the human tyrosine promoter. HEK293 cells were transfected with PSF and HDAC1 as in FIG. 5A, and total HDAC1 was immunoprecipitated, and the immuno-complex was processed for ChIP assays using primers specifically for the human tyrosine hydroxylase and GAPDH promoters. FIG. 5C provides two Western blots showing that DJ-1 disrupts the binding between the WT PSF and HDAC1. Non-denaturing cell lysates from the Hela cells co-transfected with a vector or wildtype DJ-1 (20 μg), and Flag-tagged WT PSF (10 μg) and Flag-tagged HDAC1 (10 μg) were used to immunoprecipitate total PSF. Immunoprecipitated PSF and PSF-associated HDAC1 was determined by immunoblotting with an anti-PSF and anti-Flag antibody, respectively (left panels). The expression of transfected plasmids was confirmed by western blots (anti-Flag-tag, anti-DJ-1) of the total lysates (light panels). Similar results were observed in HEK293 cells with identical experimental conditions. HeLa and HEK293 cells were used in co-immunoprecipitation experiments due to the high transfection efficiency in these cells.

FIGS. 6A and 6B show that DJ-1 inactivation leads to decreased acetylation of the human tyrosine hydroxylase promoter-bound histones. FIG. 6A shows ChIP assays of acetylated histones bound to the human tyrosine hydroxylase promoter. Various acetylated histone species from CHP-212 cells transfected with control or DJ-1 RNAi for 4 days were immunoprecipitated with specific antibodies, and amplified with primers specifically for the human tyrosine hydroxylase promoter using semi-quantitative PCR. Reactions were stopped at indicated PCR cycles for gel analysis. Input: 0.5% of input DNA before IP. The inhibition of endogenous tyrosine hydroxylase and DJ-1 by the DJ-1-specific siRNA for the ChIP assays was confirmed by Western blots. FIG. 6B shows the restoration of tyrosine hydroxylase expression by the HDAC inhibitor sodium butyrate (NaButy) in cells transfected with DJ-1 RNAi. S hours after the transfection of control (CTR) or DJ-1 RNAi constructs, CHP-212 cells were treated with increasing amount of sodium butyrate for additional 88 hours before harvesting, with 2 changes of fresh medium containing sodium butyrate during the course of the experiment. The optimal dosage of sodium butyrate was determined empirically to achieve minimal cellular toxicity, and was comparable to the tolerable dosages tested in vivo. The increased histone acetylation caused by sodium butyrate was confirmed by western blotting (data not shown). The protein levels of TH, DJ-1 and β-actin were determined by western blotting.

FIGS. 7A and 7B show that PSF sensitizes SH-SY5Y cells to dopamine-induced cell death, which is blocked by DJ-1. FIG. 7A presents six micrographs showing immunofluorescence present in dopamine-treated SH-SY5Y cells after co-transfection with GFP, PSF, or PSF and Myc-His-tagged wild-type DJ-1 expression vectors. SH-SY5Y cells transiently co-transfected with GFP or PSF with either empty vector or various DJ-1 constructs were treated with dopamine (200 μM). Dopamine treatment was initiated twenty-four hours after transfection and continued for another twenty-four hours. Cells were fixed and labeled for GFP (top panel), PSF (middle panel), or PSF and DJ-1 (bottom panel) and Hoechst. Right panels are Hoechst only images. Single arrows indicate PSF-transfected cells with apoptotic nuclear morphology, and arrowheads and double arrows indicate GFP and DJ-1/PSF transfected cells with normal nuclear morphology, respectively. FIG. 7B is a graph that quantifies apoptosis in cells expressing GFP, PSF or both DJ-1 (WT or indicated mutants) and PSF before and after treatment with dopamine. Values represent the mean±S.E.M (n-=3 with at least 150 cells per condition). P<0.01 as indicated for DJ-1 and PSF expressing cells relative to cells expressing PSF alone (column 4). *: P<0.05 relative to cells transfected with PSF and wild-type DJ-1 (column 5) by ANOVA with post-hoc Student-Neumann-Kiels test. The amount of transfected PSF remained constant in cells co-transfected with various DJ-1 mutants, ruling out the possible artifacts introduced by co-transfection.

FIG. 8 shows that DJ-1 protected against A30P α-synuclein toxicity in SH-SY5Y cells. Cultured SH-SY5Y cells were co-transfected with A30P α-synuclein and control, wild type DJ-1 (WtDJ-1), or mutant DJ-1 expression vectors. Cells were triple labeled for α-synuclein, DJ-1 and Hoechst and analyzed for apoptosis. Values represent the mean±S.E.M. n=3 experiments for Control, WtDJ-1 and L166PDJ-1, and n=2 experiments for the other DJ-1 mutants. For each experiment, at least 150 cells were scored for each condition. P<0.01 as indicated for WtDJ-1 relative to control. *P<0.05 for the indicated DJ-1 mutants relative to WtDJ-1 by ANOVA with post-hoc Student-Neumann-Kiels test.

FIG. 9 is a graph showing a quantitative analysis of apoptosis induced by the WT or mutant K338A and 1337A PSF. SH-SY5Y cells transfected with 0.5 μg of control or indicated Flag-tagged PSF constructs were fixed and labeled 4S hours post-transfection for apoptosis analysis as described (Xu, Human Mol Genetics 2005). n=3, with more than 100 cells scored per condition. *: P<0.01 relative to control.

### DETAILED DESCRIPTION OF THE INVENTION

### Definitions

By “pyrimidine tract-binding protein associated splicing factor (PSF)” is meant a protein or fragment thereof having substantial identity to the amino acid sequence provided at GenBank Accession No. P23246 and having a biological activity of PSF.

By “pyrimidine tract-binding protein associated splicing factor (PSF) biological activity” is meant having a selective interaction with DJ-1, or transcriptional regulation of a downstream gene, such as tyrosine hydroxylase.

By “huntingtin polypeptide” is meant a polypeptide or fragment thereof having substantial identity to NP_{—}002102 that binds an antibody that recognizes an anti-beta amyloid precursor polypeptide.

By “beta amyloid precursor polypeptide” is meant a protein or fragment thereof having substantial identity to GenBank Accession No: AAB20156 that binds an antibody that recognizes an anti-beta amyloid precursor polypeptide.

By “reference” is meant a standard or control condition.

By “an effective amount” is meant the amount of a required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

By “polypeptide” is meant any chain of amino acids, regardless of length or post-translational modification.

By “promoter” is meant a polynucleotide sufficient to direct transcription.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “subject” is meant a mammal, including, but not limited to, a human or non-human mammal, such as a bovine, equine, canine, ovine, or feline.

By “reporter” is meant a detectable moiety. Exemplary detectable moieties are detectable via spectroscopic, photochemical, biochemical, immunochemical, or chemical means. Suitable detectable moieties include radioactive isotopes, magnetic beads, metallic beads, colloidal particles, fluorescent dyes, electron-dense reagents, enzymes (for example, as commonly used in an ELISA), biotin, digoxigenin, or haptens.

By “tyrosine hydroxylase” is meant having substantial identity to the protein sequence provided at GenBank Accession No. NP_{—}954986, NP_{—}000351, or NP_{—}954987. and having dihydroxyphenylalanine synthesizing activity.

By “apoptosis” is meant the process of cell death wherein a dying cell displays a set of well-characterized biochemical hallmarks that include cell membrane blebbing, cell soma shrinkage, chromatin condensation, and/or DNA laddering.

By “neurodegenerative disease” is meant any disorder characterized by excess neuronal cell death. Exemplary neurodegenerative diseases include Parkinson's disease, Huntington's disease, Alzheimer's disease, Kennedy's Disease, and spinocerebellar ataxia.

By “sumoylation” is meant the post-translational modification of a polypeptide by the addition of a SUMO peptide. Sumoylation is described, for example, by Johnson et al., Annu. Rev. Biochem. 73:355-82, 2004. The sequence of SUMO peptides SUMO-1, SUMO-2, and SUMO-3 is found at GenBank Accession No. NP_{—}003343, NP_{—}001005849, and NP_{—}008867, respectively.

By “sumoylation substrate” is meant any polypeptide or fragment thereof capable of being modified by the addition of a SUMO peptide.

By “enhances” is meant a positive alteration of at least 10%, 15%,25%, 50%, 75%, or 100%.

By “fragment” is meant a portion of a protein or nucleic acid that is substantially identical to a reference protein or nucleic acid (e.g., one of those listed in Tables 1 or 2), and retains at least 50% or 75%, more preferably 80%, 90%, or 95%, or even 99% of the biological activity of the reference protein or nucleic acid using a nematode bioassay as described herein or a standard biochemical or enzymatic assay.

As used herein, “obtaining,” as in “obtaining an agent” includes synthesizing, purchasing or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “ameliorate” is meant decrease, suppress, attenuate, diminish, arrest, or stabilize the development or progression of a disease.

By “compound” is meant a small molecule, nucleic acid molecule, polypeptide or fragment thereof, or any other substance that has the potential of affecting the function of an organism. Such a compound may be, for example, a naturally occurring, semi-synthetic, or synthetic agent. For example, the candidate compound may be a drug that targets a specific function of an organism. A candidate compound may also be an antibiotic or a nutrient. A therapeutic compound may decrease, suppress, attenuate, diminish, arrest, or stabilize the development or progression of disease or disorder in a eukaryotic organism.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “transformed cell” is meant a cell into which (or into an ancestor of which) has been introduced, by means of recombinant DNA techniques, a polynucleotide molecule encoding (as used herein) a polypeptide of the invention.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

### METHODS OF THE INVENTION

The invention generally provides screening methods for the identification of therapeutic compounds useful for the treatment of Parkinson's disease, and related prophylactic and therapeutic compositions and methods. The invention is based, in part, on the observation that DJ-1 and a DJ-1 interacting protein, pyrimidine tract-binding protein associated splicing factor (PSF), transcriptionally regulate tyrosine hydroxylase, a biosynthetic enzyme that is required for dopamine synthesis.

### Dopamine Biosynthesis

Dopamine is a biogenic amine neurotransmitter that is derived from the amino acid tyrosine. The first step in dopamine synthesis is catalyzed by the rate-limiting enzyme tyrosine hydroxylase in a reaction requiring oxygen as a co-substrate and tetrahydrobiopterin as a cofactor to synthesize dihydroxyphenylalanine (DOPA). DOPA is subsequently decarboxylated by DOPA decarboxylase to produce dopamine.

The major dopamine-containing area of the brain is the corpus striatum, which receives major input from the substantia nigra and plays an essential role in the coordination of body movements. In Parkinson's disease the dopaminergic neurons of the substantia nigra degenerate, leading to a characteristic motor dysfunction. Although dopamine does not readily cross the blood-brain barrier, its precursor, levodopa, does. Therefore, the disease can be treated by administering levodopa together with carbidopa, a dopamine decarboxylase inhibitor, and selegiline, a monoamine oxidase inhibitor. While this treatment can alleviate some of the symptoms of Parkinson's disease, it cannot stop the degeneration of the dopaminergic neurons underlying the disorder. Therapeutic methods that prevent, slow, or stabilize the death of these neurons are required.

### DJ-1, Transcriptional Regulation and Parkinson's Disease

Genetic evidence links the loss of DJ-1 function to early-onset Parkinsonism¹. DJ-1 is highly conserved throughout evolution and has been shown to regulate oxidative stress, apoptosis, protein aggregation and transcription in various subcellular compartments²⁻⁷. In addition, DJ-1 shares structural similarity with a bacterial protease and harbors a catalytic cysteine⁸⁻¹⁰, suggesting that DJ-1 is a cellular cysteine protease. In vitro experiments have also demonstrated that DJ-1 has neuroprotective activity^{3,6,7,8}. Nevertheless, it remains unclear why the loss of DJ-1 function contributes to the selective loss of dopaminergic functions.

Before DJ-1 was linked to familial Parkinson's disease, DJ-1 was known to modulate androgen receptor function in the testis. DJ-1 interacts with the transcriptional repressors PIASxa and DJBP and relieves androgen receptor transcriptional inhibition^{9,10}. PIASxa is a small ubiquitin-like modifier (SUMO) E3 ligase mediating the covalent coupling of SUMO proteins (sumoylation) to multiple transcriptional factors, including androgen receptor^{11,12}, Sumoylation is a reversible ATP-dependant process similar to ubiquitination, and requires the participation of E1 activating enzymes, E2 conjugating enzymes and E3 ligases¹¹⁻¹³. While ubiquitination regulates protein stability, sumoylation has been shown to modulate the subcellular localization and transcriptional activity of the substrates^{11,13,14}. Besides PIAS proteins, DJ-1 interacts with Sumo-1, SUMO activating enzyme Uba2, and conjugating enzyme ubc-9 in yeast two-hybrid systems^{7,15}. In addition, in cells overexpressing multiple components of the sumoylation machinery, DJ-1 is itself sumoylated¹⁵.

The role of transcriptional dysregulation in the pathogenesis of neurodegenerative diseases is manifested by the molecular mechanisms of polyglutamine diseases^{16,17}. Pathogenic polyglutamine expansion in huntingtin, androgen receptor, and various spinocerebellar ataxia proteins results in aberrant protein interactions and in the inhibition of histone acetyltransferase activity, thus affecting gene transcription^{16,17}. Interestingly, sumoylation of huntingtin promotes its ability to repress transcription and exacerbates neurodegeneration in a Drosophila model of Huntington's disease¹⁸. Nevertheless, the relationship between transcriptional regulation and Parkinson's disease is unclear. The transcriptional regulators PSF and p54nrb are the major binding partners of DJ-1 in human dopaminergic cells⁶. Further, DJ-1 prevents PSF-mediated transcriptional repression and apoptosis as a transcriptional co-activator.

As reported in more detail below, DJ-1 and PSF transcriptionally regulate human tyrosine hydroxylase. DJ-1 promotes tyrosine hydroxylase expression by inhibiting PSF sumoylation and increasing histone acetylation. These results indicate that transcriptional dysregulation caused by DJ-1 inactivation is a molecular mechanism underlying the selective vulnerability of the dopaminergic pathway in Parkinson's disease, and suggests that compounds that modulate PSF sumoylation are likely to be useful for the treatment of Parkinson's disease.

### Polypyrimidine Tract-Binding Protein-Associated Splicing Factor

Polypyrimidine tract-binding protein-associated splicing factor (PSF), which binds the polypyrimidine tract of mammalian introns, is an essential pre-mRNA splicing factor. PSF, which is required early in spliceosome formation (Patton et al., Genes Dev. 7: 393-406, 1993) associates with DJ-1. As reported herein, PSF acts as a transcriptional repressor in dopaminergic cells where it also promotes cell death. Results described below indicated that PSF transcriptional repression depends on a post-translation modification of PSF, known as sumoylation. DJ-1 blocked PSF-induced transcriptional repression and cell death. Consistent with this observation, the expression of human tyrosine hydroxylase, the rate-limiting enzyme for dopamine synthesis, was repressed by sumoylated PSF and activated by DJ-1

The amino acid sequence of PSF, which is encoded by the nucleic acid sequence provided at GenBank Accession No. BC004534, is shown below in Table 1. This amino acid sequence corresponds to GenBank Accession No. P23246.

TABLE 1

```
    1 MSRDRFRSRG GGGGGFHRRG GGGGRGGLHD FRSPPPGMGL NQNRGPMGPG PGQSGPKPPI
  61 PPPPPHQQQQ QPPPQQPPPQ QPPPHQPPPH PQPHQQQQPP PPPQDSSKPV VAQGPGPAPG
121 VGSAPPASSS APPATPPTSG APPGSGPGPT PTPPPAVTSA PPGAPPPTPP SSGVPTTPPQ
181 AGGPPPPPAA VPGPGPGPKQ GPGPGGPKGG KMPGGPKPGG GPGLSTPGGH PKPPHRGGGE
241 PRGGRQHHPP YHQQHHQGPP PGGPGGRSEE KISDSEGFKA NLSLLRRPGE KTYTQRCRLF
301 VGNLPADITE DEFKRLFAKY GEPGEVFINK GKGFGFIKLE SRALAEIAKA ELDDTPMRGR
361 QLRVRFATHA AALSVRNLSP YVSNELLEEA FSQFGPIERA VVIVDDRGRS TGKGIVEFAS
421 KPAARKAFER CSEGVFLLTT TPRPVIVEPL EQLDDEDGLP EKLAQKNPMY QKERETPPRF
481 AQHGTFEYEY SQRWKSLDEM EKQQREQVEK NMKDAKDKLE SEMEDAYHEH QANLLRQDLM
541 RRQEELRRME ELHNQEMQKR KEMQLRQEEE RRRREEEMMI RQREMEEQMR RQREESYSRM
601 GYMDPRERDM RMGGGGAMNM GDPYGSGGQK FPPLGGGGGI GYEANPGVPP ATMSGSMMGS
661 DMRTERFGQG GAGPVGGQGP RGMGPGTPAG YGRGREEYEG PNKKPRF
```

### Regulation of Transcription and Apoptosis by DJ-1 and PSF

Before DJ-1 was linked to Parkinson's disease, it was shown that DJ-1 promotes the transcriptional activity of androgen receptor by antagonizing two repressors highly expressed in the testis, protein inhibitor of activated STATx alpha (PIASxa) and DJ-1-binding protein^{9,10}. PIASxa is a SUMO ligase and represses the activity of transcription factors including signal transducer and activator of transcription proteins (STAT) and AR by facilitating SUMO-1 conjugation^{10,19}. DJ-1 blocks PIASxa-mediated transcriptional repression by preventing its binding to androgen receptor¹⁰. Meanwhile, DJ-1 disrupts the recruitment of the histone deacetylase repressor complexes by DJ-1-binding protein and restores transcriptional activation by androgen receptor. To investigate the role of DJ-1 in Parkinson's disease pathogenesis, an unbiased proteomic approach was used to identify DJ-1-interacting proteins in the human dopaminergic cells⁶. Mass spectrometry analysis revealed that the major DJ-1-interacting proteins in these cells were p54nrb and pyrimidine-tract binding protein-associated splicing factor (PSF).

p54nrb and PSF are multi-functional nuclear proteins²⁰. PSF was originally identified as a protein interacting with polypyrimidine tract²¹, an intronic region important for splicing. In addition, PSF is a part of the spliceosome C complex²² and required for in vitro splicing of pre-mRNA²¹. Both p54nrb and PSF contain homologous RNA recognition motifs, and form heterodimeric complex capable of binding RNA²⁰. p54nrb and PSF heterodimers also bind DNA and regulate gene transcription^{20,23,24}. More specifically, PSF has been shown to recruit the HDAC repressor complex and cause transcriptional repression²⁴. Using a transcription reporter system, DJ-1 was found to cooperate with p54nrb and inhibit the transcriptional silencing activity of PSF⁶. Moreover, DJ-1 and p54nrb prevented apoptosis induced by PSF, α-synuclein or oxidative stress. Parkinson's Disease-associated DJ-1 mutants show decreased nuclear localization and significantly reduced transcriptional activation and protection against apoptosis⁶.

As reported herein, DJ-1 and PSF directly bind and transcriptionally regulate the human tyrosine hydroxylase (TH) promoter. PSF repressed, while DJ-1 activated, tyrosine hydroxylase expression. Moreover, the silencing of the tyrosine hydroxylase promoter by PSF required sumoylation, a process inhibited by the wild-type DJ-1, but not the pathogenic DJ-1 mutants. This observation indicated the functional relevance of PSF sumoylation to Parkinson's disease pathogenesis. Since SUMO-deficient PSF mutants failed to induce apoptosis as efficiently as wild-type PSF, neuronal apoptosis, like tyrosine hydroxylase expression, is at least partially affected by PSF sumoylation and DJ-1-mediated transcriptional regulation in dopaminergic cells. Therefore, by modulating sumoylation and mimicking DJ-1 functions, both neuronal survival and nigralstriatal functions were regulated.

### Sumoylation, Acetylation, DJ-1 and Neurodegenerative Diseases

The small ubiquitin-related modifiers (SUMO) covalently modify a number of proteins and share structural homology with ubiquitin, the central component in the proteasome degradation machinery^{11,13}. Four highly homologous SUMO molecules have been described, and may contribute to functional redundancy and tissue-specific activity^{11,13}. Like ubiquitination, sumoylation is a form of dynamic and reversible post-translational modification, and is characterized by the formation of an iso-peptide bond between a C-terminal glycine in the SUMO molecule and a lysine residue in the protein substrate^{11,14}, sumoylation typically occurs at a consensus tetra-peptide site consisting ψKxE, where ψ_{.} is a large hydrophobic residue, and x is any amino acid. Sumoylation is an ATP-dependent reaction that requires the participation of the E1 activating enzymes, E2 conjugating enzymes, and E3 ligase. Three SUMO E3 ligases, RanBP2, PIAS (protein inhibitor of activated STAT) proteins, and polycomb group protein Pc2, have been identified and serve as adaptors to facilitate the transfer of SUMO proteins from the only E2 conjugating enzyme described, Ubc9, to various substrates. Most of the SUMO substrates are nuclear proteins regulating transcription, chromatin structure, and signal transduction, and DNA repair¹¹⁻¹⁴. While ubiquitination primarily regulates substrate stability, sumoylation has been shown to alter the subcellular or subnuclear distribution and the activities of the substrates¹¹⁻¹⁴.

The link between DJ-1 and SUMO was demonstrated by the interactions between DJ-1 and the components of the SUMO machinery, including two SUMO E3 ligases, PIASxa and PIASy¹⁰, E2 Ubc 9 and SUMO-1^{7,15}, using the yeast two-hybrid system. As reported herein, DJ-1 suppressed sumoylation of PSF and affected the subnuclear localization of PSF. Furthermore, DJ-1 markedly decreased overall abundance of the SUMO-1-conjugated proteins. Therefore, DJ-1 serves as an inhibitor of sumoylation.

In most cases, SUMO modification of transcriptional regulators results in the repression of affected promoters^{13,14}. Sumoylation is thought to promote the recruitment of blown general transcriptional repressor complexes, such as the hi stone deacetylase complex^{13,25}. The acetylation of nucleasomal histones facilitates the unwinding of chromatin and promotes transcription²⁶. In addition, increased acetylation of many transcription factors leads to enhanced transcriptional activities²⁷. By removing the acetyl groups from nuclear histones and transcriptional factors, HDACs stimulate gene silencing. Therefore, sumoylation and acetylation are coupled molecular switches to control gene expression that are likely controlled by DJ-1. As reported herein, inactivation of DJ-1 resulted in decreased acetylation of all four nucleasomal histones associated with the human tyrosine hydroxylase promoter. Since PSF is known to recruit HDAC complexes and repress transcription²⁴, DJ-1 likely promotes histone acetylation and gene expression by preventing the sumoylation of PSF and the subsequent recruitment of the repressor complex. Disruptions in DJ-1 function, therefore, likely lead to transcriptional dysfunction.

Transcriptional dysfunction is thought to be one cause of neurodegenerative disease, such as neurodegenerative disease related to polyglutamine expansion^{16,17} Pathogenic polyglutamine expansions in huntingtin, androgen receptor, and various spinocerebellar ataxia proteins results in aberrant protein interactions and the inhibition of histone acetyltransferase activity, thus affecting gene transcription^{16,17}.

### Inhibition of PSF Sumoylation by DJ-1 as a Therapeutic Approach for Parkinson's Disease

Sumoylation and transcriptional control by DJ-1 is relevant to Parkinson's disease pathogenesis and is involved in pathways affecting both dopaminergic neuronal survival and function. Loss-of-function mutations in DJ-1 cause early onset of Parkinson's disease. As reported herein, DJ-1, PSF and sumoylation are involved in the regulation of both nigralstriatal and apoptosis pathways in dopaminergic cells. Wild-type DJ-1 inhibited PSF sumoylation, while several disease-causing mutations in DJ-1 disrupted this capability. It is, therefore, likely that the regulation of sumoylation by DJ-1 is linked to Parkinson's disease pathogenesis. In addition, the multiple reported functions of DJ-1 are consistent with the ability of DJ-1 to regulate gene expression.

Although DJ-1 is a multifunctional protein with a number of reported interacting partners^{7,9,10,15}, DJ-1 appears to bind and regulate different sets of proteins in different tissues. In human dopaminergic cells, the type of cells affected in Parkinson's disease patients, PSF and its tightly associated p54nrb are the major DJ-1-binding proteins⁶. This protein complex has not been reported in other cell types or tissues, or in the yeast two-hybrid systems.

### Screens for PSF Sumoylation Modulators

As reported herein, sumoylated PSF represses the expression of tyrosine hydroxylase. DJ-1 relieved this repression by decreasing PSF sumoylation. In addition, PSF overexpression induced neuronal apoptosis and DJ-1 prevented PSF-mediated cell death. Therefore, PSF and DJ-1 likely regulate multiple genes important for the survival and normal functioning of dopaminergic neurons. Based on these observations, it is likely that polypeptide sumoylation serves as a common mechanism for regulating the activity of polypeptides or the expression of genes required for neural maintenance or function. Accordingly, compositions that modulate the sumoylation of a polypeptide associated with a neurodegenerative disorder are useful for the treatment of a neurodegenerative disease. While the examples below relate to screens for modulators of PSF sumoylation, PSF is merely an exemplary sumoylation substrate. Virtually any other sumoylated polypeptide associated with neuronal cell death or a neurodegenerative disease (e.g., huntingtin (GenBank Accession No: NP_{—}002102), androgen receptor (GenBank Accession No: AAD14959), beta amyloid precursor peptide (GenBank Accession No: AAB20156) could be substituted for PSF in the screens described herein.

One skilled in the art will appreciate that any method for detecting a reduction in the level of sumoylated PSF known in the art may be used in the methods of the invention. In one example, candidate compounds are added at varying concentrations to the culture medium of a cell (e.g., a dopaminergic neuron). The contacted cell is lysed and the PSF protein present in the lysate is isolated, for example, by contacting the lysate with an anti-PSF antibody fixed to a solid substrate. The level of sumoylated PSF is then detected, for example, by contacting the isolated PSF protein with an anti-Sumo antibody. Desirably, the anti-Sumo antibody is linked to a detectable reporter, such as a fluorescent marker. The level of sumoylated PSF present in the cell contacted with the candidate compound is compared to the level present in a control cell not contacted with the candidate compound. Sumoylation is a dynamic change that varies as cellular conditions change. The removal of SUMO from its substrate is mediated by SUMO-cleaving enzymes, also known as isopeptidases. All known SUMO-cleaving enzymes have a 200 amino acid C-terminal domain, the U1p domain, that has SUMO cleaving activity. Compounds that enhance the reversal of PSF sumoylation by increasing SUMO cleavage, for example, can also be identified using this in vitro assays. A candidate compound that decreases the sumoylation of PSF is identified as useful for the treatment of a neurodegenerative disorder.

The invention further provides sumoylation responsive gene constructs comprising a promoter whose expression is regulated by a transcription factor subject to sumoylation operably linked to a detectable reporter and methods of using such constructs to identify compounds that alter the sumoylation status of the transcription factor. In one embodiment, a tyrosine hydroxylase promoter, or a fragment thereof (e.g., the promoter), is operably linked to a detectable reporter (e.g., horseradish peroxidase, alkaline phosphatase, luciferase, GFP) and the construct is expressed in a mammalian cell that expresses PSF. In the case of the tyrosine hydroxylase promoter, expression of the reporter construct is repressed in the presence of sumoylated PSF. Compounds that relieve this repression are identified by an increase in the expression of the detectable reporter. Such compounds decrease PSF sumoylation and/or increase tyrosine hydroxylase expression, and are useful for the treatment or prevention of neurodegenerative diseases, including Parkinson's disease.

Compounds that inhibit sumoylation include those that bind to a PSF sumoylation consensus sequence and physically block sumoylation. Sumoylation consensus sequences are known in the art (see, for example, Johnson, Annu. Rev. Biochem. 73:355-82, 2004), and include the sequence _{ψ}KXD, where _{ψ} is any hydrophobic amino acid (e.g., leucine, isoleucine, valine or proline), K is the lysine residue that is modified, X is any amino acid, and D is a glutamic acid. Methods of identifying compounds that bind to a particular amino acid sequence are known to the skilled artisan and are described herein. In one embodiment, a protein (e.g., PSF) that includes a sumoylation consensus sequence, or a fragment thereof, is fixed to a solid substrate. The protein is then contacted with a candidate compound under conditions that permit binding. The protein-candidate compound complex is then washed to eliminate compounds that bind non-specifically. Candidate compounds that specifically bind a sumoylation consensus sequence are subsequently isolated. Such compounds are expected to block sumoylation and are useful for treating a disease or disorder associated with increased levels of sumoylation, such as a neurodegenerative disorder (e.g., Parkinson's disease).

Compounds that inhibit PSF sumoylation can also be identified using in vitro assays. In one embodiment, a PSF polypeptide is incubated in vitro in the presence of a candidate compound under conditions that permit sumoylation. In vitro methods of assaying sumoylation are known in the art; see, for example, Pichler et al., Cell 108: 109-120, 2002. In brief, a sumoylation substrate, such as a peptide containing a sumoylation consensus sequence, a PSF polypeptide, or a fragment thereof, is immobilized in assay plates and incubated in the presence of a SUMO activating enzyme (E1), a SUMO conjugating enzyme (E2 or Ubc9), and a SUMO-protein ligase (E3s), under conditions that permit sumoylation in the presence or the absence of a candidate compound. The level of PSF sumoylation in the presence of the candidate compound is compared to the level of PSF sumoylation in the absence of the candidate compound. A compound that reduces PSF sumoylation is identified as useful in the methods of the invention. In one embodiment, a compound that reduces sumoylation is a peptide that competes for sumoylation with a naturally occurring sumoylation consensus sequence. In various embodiments, the peptide is a 5, 10, 15, 20, 25, 30, 35, 40, 45, or 50 amino acid fragment of a sumoylation substrate (e.g., PSF, amyloid polypeptide, androgen receptor, huntingtin).

The present screening methods are easily adapted for the identification of other neurodegenerative diseases related to sumoylation (e.g., Huntington's disease, Kennedy's disease, and Alzheimer's disease). While PSF is specifically described, one skilled in the art appreciates that any sumoylated protein associated with a neurodegenerative disease (e.g., huntingtin, androgen receptor, and amyloid precursor protein) may be used in the methods described herein. In addition to provide methods of screening for prophylactic or therapeutic compounds for the treatment of a neurodegenerative disease, the methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Screens for Compounds that Enhance Cell Survival

Compounds that alter sumoylation are likely to enhance the survival of neuronal cells at risk of undergoing apoptosis. Based in part on this discovery, compositions of the invention are useful for identifying compounds that alter the sumoylation of a neuronal polypeptide expressed by a cell at risk of apoptosis or by modulating the activity of a SUMO activating enzyme (E1), a SUMO conjugating enzyme (E2 or Ubc9), or a SUMO-protein ligase (E3s) that mediates the sumoylation of a polypeptide expressed in a neuronal cell at risk of apoptosis. If desired, compounds that modulate sumoylation are tested for efficacy in reducing cell death in a cell at risk thereof. In one embodiment, tissues or cells treated with a candidate compound that modulates sumoylation are compared to untreated control samples to identify therapeutic agents that enhance cell survival or reduce cell death in a cell at risk thereof. Any number of methods are available for carrying out screening assays to identify new candidate compounds that alter sumoylation and enhance neuronal survival.

In one working example, candidate compounds (e.g., compounds that alter sumoylation) are added at varying concentrations to the culture medium of cultured cells (e.g., neuronal cultures) prior to, concurrent with, or following the addition of a proapoptotic agent. Cell survival is then measured using standard methods. The level of apoptosis in the presence of the candidate compound is compared to the level measured in a control culture medium lacking the candidate molecule. A compound that promotes an increase in cell survival, a reduction in apoptosis, or an increase in cell proliferation is considered useful in the invention; such a candidate compound may be used, for example, as a therapeutic to prevent, delay, ameliorate, stabilize, or treat the toxic effects of a neurodegenerative disease. In other embodiments, the candidate compound prevents, delays, ameliorates, stabilizes, or treats a disease or disorder characterized by excess cell death (e.g., a neurodegenerative disorder) or promotes the survival of a neuronal cell at risk of cell death. Such therapeutic compounds are useful in vivo as well as ex vivo.

In yet another example, candidate compounds are screened for those that specifically bind to a polypeptide involved in sumoylation (e.g., SUMO activating enzyme (E1), a SUMO conjugating enzyme (E2 or Ubc9), a SUMO-protein ligase (E3s)), or to a sumoylation substrate expressed by a cell at risk of apoptosis. The efficacy of such a candidate compound is dependent upon its ability to interact with at least one of these polypeptides. Such an interaction can be readily assayed using any number of standard binding techniques and functional assays (e.g., those described in Ausubel et al., supra). In one embodiment, the compound is assayed in a cell in vitro for binding to a polypeptide involved in sumoylation or to a sumoylation substrate and for the promotion of cell survival. In one particular working example, a candidate compound that binds to a polypeptide involved in sumoylation or to a sumoylation substrate is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound that binds specifically to a polypeptide involved in sumoylation or to a sumoylation substrate is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds and identified using such methods are then assayed for their effect on cell survival as described herein.

In another example, a candidate compound is coupled to a radioisotope or enzymatic label such that binding of a candidate compound to a sumoylation substrate (e.g. a sumoylation substrate expressed in a neuronal cell at risk of apoptosis) can be determined by detecting the labeled compound and the substrate in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a sumoylation substrate (e.g., a sumoylated polypeptide expressed in a neuronal cell at risk of apoptosis) or a sumoylated portion thereof is contacted with a test compound and the ability of the test compound to bind to the polypeptide is evaluated.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a polypeptide involved in sumoylation or a sumoylation substrate, can be accomplished using real-time Biomolecular Interaction Analysis (BIA) (see, e.g., Sjolander, S. and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

It may be desirable to immobilize either the candidate compound or the sumoylation substrate to facilitate separation of complexed from uncomplexed forms of one or both of the proteins, as well as to accommodate automation of the assay. Binding of a candidate compound to a sumoylation substrate can be accomplished in any vessel suitable for containing the reactants. Examples of such vessels include microtiter plates, test tubes, and micro-centrifuge tubes. In one embodiment, a fusion protein can be provided which adds a domain that allows a sumoylation substrate to be bound to a matrix. For example, glutathione-S-transferase polypeptide fusion proteins can be adsorbed onto glutathione sepharose beads (Sigma Chemical, St. Louis, Mo.) or glutathione derivatized microtiter plates, which are then combined with a test compound, and the mixture incubated under conditions conducive to complex formation (e.g., at physiological conditions for salt and pH). Following incubation, the beads or microtiter plate wells are washed to remove any unbound components, the matrix immobilized in the case of beads, and complex formation is determined either directly or indirectly, for example, as described above.

Other techniques for immobilizing a complex of a test compound and a sumoylation substrate on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, an antibody is identified that reacts with an epitope on the sumoylation substrate. Methods for detecting binding of an anti-sumoylation substrate antibody are known in the art and include immunodetection of complexes, as well as enzyme-linked assays which rely on detecting an enzymatic activity associated with the channel. Antibodies that bind a sumoylation substrate are then tested for the ability to block sumoylation. Such antibodies may also be tested for cell survival promoting activity as described herein.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D.S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the sumoylation substrate, e.g., by including a membrane component or synthetic membrane components.

Compounds isolated by this method (or any other appropriate method) may, if desired, be further purified (e.g., by high performance liquid chromatography). In addition, these candidate compounds may be tested for their ability to modulate sumoylation in a neuronal cell at risk of cell death, to reduce cell death, or to promote cell survival. Compounds isolated by this approach may also be used, for example, as therapeutics to treat a neurodegenerative disease in a subject. Compounds that are identified as binding to a sumoylation substrate or a polypeptide involved in sumoylation with an affinity constant less than or equal to 10 mM are considered particularly useful in the invention. Alternatively, any in vivo protein interaction detection system, for example, any two-hybrid assay may be utilized.

One skilled in the art appreciates that the effects of a candidate compound on sumoylation are typically compared to the sumoylation of the substrate in the absence of the candidate compound. Thus, the screening methods include comparing the value of a cell modulated by a candidate compound to a reference value of an untreated control cell.

Molecules that increase sumoylation include organic molecules, peptides, peptide mimetics, polypeptides, and nucleic acids that bind to a sumoylation substrate encoding nucleic acid sequence or a sumoylation substrate and increase its expression or biological activity are preferred.

Each of the sequences listed herein may also be used in the discovery and development of a therapeutic compound for the treatment of a neurodegenerative disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra). Small molecules of the invention preferably have a molecular weight below 2,000 daltons, more preferably between 300 and 1,000 daltons, and most preferably between 400 and 700 daltons. It is preferred that these small molecules are organic molecules.

The invention also includes novel compounds identified by the above-described screening assays. Optionally, such compounds are characterized in one or more appropriate animal models to determine the efficacy of the compound for the treatment of a neurodegenerative disease. Desirably, characterization in an animal model can also be used to determine the toxicity, side effects, or mechanism of action of treatment with such a compound. Furthermore, novel compounds identified in any of the above-described screening assays may be used for the treatment of a neurodegenerative disease in a subject. Such compounds are useful alone or in combination with other conventional therapies known in the art.

### Cells for Use in Screens

The invention is flexible and can be used to screen with a wide variety of cells and cell lines that have been transformed by one or a combination of the expression vectors provided herein. Suitable cells and cell lines are generally eukaryotic and can be transformed by the expression vector. A number of types of cells may act as suitable host cells for the expression vector.

In one approach, the screens described herein are carried out in dopaminergic cells having neuronal characteristics. Such cells are known in the art and include, for example, BE(2)-M17 neuroblastoma cells (Martin et al., J. Neurochem. 2003 November; 87(3):620-30), Cath.a-differentiated (CAD) cells (Arboleda et al., J Mol Neurosci. 2005; 27(1):65-78), CSM14.1 (Haas et al., J Anat. 2002 July; 201(1):61-9), MN9D (Chen et al., Neurobiol Dis. 2005 August; 19(3):419-26), N27 cells (Kaul et al., J Biol Chem. 2005 Aug. 5; 280(31):28721-30), PC12 (Gorman et al., Biochem Biophys Res Commun. 2005 Feb. 18; 327(3):S01-10), SN4741 (Nair et al., Biochem J. 2003 Jul. 1; 373(Pt 1):25-32), CHP-212, SH-SY5Y, and SK-N-BE.

The invention is not limited to neuronal cell lines, however. Other mammalian host cells useful in the methods of the invention include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

The following Tables II-VI provide illustrative cells for use with the invention that can be obtained from the ATCC. In particular, Table II provides illustrative non-tumor, neuronal-like cells; tumor-derived neuronal-like cells, glioblastoma cells, medulloblastome-derived cells; retinoblastoma-derived cells; and neuroendocrine tissue; Table III provides exemplary tumor cell lines; Table IV provides various mammary gland derived cells lines; Table V provides illustrative prostate derived cells lines; and Table VI provide examples of testis-derived cell lines.

TABLE II

| ATCC No. | Species | Name | Tissue Source |
|---|---|---|---|
| CRL-10442 | human | HCN-1A | brain |
| CRL-10742 | human | HCN-2 | brain |
| CCL-127 | human | IMR-32 | brain; neuroblastoma |
| CRL-1718 | human | CCF-STTG1 | brain; astrocytoma |
| CRL-2060 | human | PFSK-1 | brain; cerebellum; |
| | | | malignant primitive |
| | | | neuroectodermal tumor |
| CRL-2137 | human | SK-N-AS | brain; neuroblastoma |
| CRL-2142 | human | SK-N-FI | brain; neuroblastoma |
| CRL-2149 | human | SK-N-DZ | brain; neuroblastoma |
| CRL-2266 | human | SH-SY5Y | brain; neuroblastoma |
| CRL-2267 | human | BE(2)-M17 | brain; neuroblastoma |
| CRL-2268 | human | BE(2)-C | brain; neuroblastoma |
| CRL-2270 | human | MC-IXC | brain; neuroblastoma |
| CRL-2271 | human | SK-N-BE(2) | brain; neuroblastoma |
| CRL-2273 | human | CHP-212 | brain; neuroblastoma |
| CRL-8621 | human | SVGp12 | brain |
| HTB-10 | human | SK-N-MC | brain; |
| | | | neuroepithelioma, |
| | | | metastic site: supra- |
| | | | orbital area |
| HTB-11 | human | SK-N-SH | brain; neuroblastoma, |
| | | | metastic site: bone |
| | | | marrow |
| HTB-12 | human | SW 1088 | brain; astrocytoma |
| HTB-13 | human | SW 1783 | brain; astrocytoma |
| HTB-15 | human | U-118 MG | brain; glioblastoma; |
| | | | astrocytoma |
| CRL-1620 | human | A172 | brain; glioblastoma |
| CRL-1690 | human | T98G | brain; glioblastoma |
| | | | multiforme |
| CRL-2020 | human | DBTRG-05MG | brain; glioblastoma |
| CRL-2365 | human | M059K | brain; malignant |
| | | | glioblastoma; glioma |
| CRL-2366 | human | M059J | brain; malignant |
| | | | glioblastoma; glioma |
| CRL-7773 | human | TE 615.T | brain; |
| | | | ganglioneuroblastoma |
| HTB-138 | human | Hs 683 | brain; glioma |
| HTB-14 | human | U-87 MG | brain; glioblastoma; |
| | | | astrocytoma |
| HTB-148 | human | H4 | brain; neuroglioma |
| HTB-16 | human | U-138 MG | brain; glioblastoma |
| CRL-8805 | human | TE671 | brain; cerebellum; |
| | | subline No. 2 | medulloblastoma |
| HTB-185 | human | D283 Med | brain; cerebellum; |
| | | | medulloblastoma, |
| | | | matastic site: |
| | | | peritoneum |
| HTB-186 | human | Daoy | brain; cerebellum; |
| | | | desmoplastic cerebellar |
| | | | medulloblastoma |
| HTB-187 | human | D341 Med | brain; cerebellum; |
| | | | medulloblastoma |
| HTB-169 | human | WERI-Rb-1 | retinoblastoma; eye; |
| | | | retina |
| HTB-18 | human | Y79 | retinoblastoma; eye; |
| | | | retina |
| CRL-5813 | human | NCI-H660 | lung; carcinoma; small |
| | | | cell lung cancer |
| | | | extrapulmonary origin |
| | | | (prostate), metastic |
| | | | site: lymph node |
| CRL-5893 | human | NCI-H1770 | lung; carcinoma; non- |
| | | | small cell lung cancer; |
| | | | metastic site: lymph |
| | | | node |
| CRL-2139 | human | SK-PN-DW | malignant primitive |
| | | | neuroectodermal |
| | | | tumor; retroperitoneal |
| | | | embryonal tumor |
| CRL-1973 | human | NTERA-2 cl.D1 | malignant pluripotent |
| | | | embryonal carcinoma; |
| | | | testis, metastic site: |
| | | | lung |

TABLE III

| ATCC No. | Name | Cancer Type | Tissue Source |
|---|---|---|---|
| CRL-7365 | Hs 605.T | carcinoma | mammary gland; breast |
| CRL-7368 | Hs 606 | carcinoma | mammary gland; breast |
| HTB-126 | Hs 578T | ductal carcinoma | mammary gland; breast |
| CRL-2320 | HCC1008 | ductal carcinoma | mammary gland; breast |
| CRL-2338 | HCC1954 | ductal carcinoma | mammary gland; breast |
| CRL-7345 | Hs 574.T | ductal carcinoma | mammary gland; breast |
| CRL-2314 | HCC38 | primary ductal | mammary gland; breast |
| | | carcinoma | |
| CRL-2321 | HCC1143 | primary ductal | mammary gland; breast |
| | | carcinoma | |
| CRL-2322 | HCC1187 | primary ductal | mammary gland; breast |
| | | carcinoma | |
| CRL-2324 | HCC1395 | primary ductal | mammary gland; breast |
| | | carcinoma | |
| CRL-2331 | HCC1599 | primary ductal | mammary gland; breast |
| | | carcinoma | |
| CRL-2336 | HCC1937 | primary ductal | mammary gland; breast |
| | | carcinoma | |
| CRL-2340 | HCC2157 | primary ductal | mammary gland; breast |
| | | carcinoma | |
| CRL-2343 | HCC2218 | primary ductal | mammary gland; breast |
| | | carcinoma | |
| CRL-7482 | Hs 742.T | scirrhous | mammary gland; breast |
| | | adenocarcinoma | |

TABLE IV

| Species | Cell Line Name | ATCC No. | Description |
|---|---|---|---|
| human | MCF 10A | CRL-10317 | fibrocystic disease |
| human | MCF 10F | CRL-10318 | fibrocystic disease |
| human | MCF-10-2A | CRL-10781 | fibrocystic disease |
| human | MCF-12A | CRL-10782 | |
| human | MCF-12F | CRL-10783 | |
| human | Hs 564(E).Mg | CRL-7329 | |
| human | Hs 565(A).Mg | CRL-7330 | cyst |
| human | Hs 565(D).Mg | CRL-7333 | cyst |
| human | Hs 579.Mg | CRL-7347 | |
| human | Hs 617.Mg | CRL-7379 | |
| human | Hs 873.T | CRL-7610 | abnormal |
| human | Hs 874.T | CRL-7611 | abnormal |
| human | Hs 875.T | CRL-7612 | abnormal |
| human | Hs 877.T | CRL-7613 | abnormal |
| human | Hs 879(B).T | CRL-7615 | |
| human | Hs 880.T | CRL-7616 | abnormal |
| human | Hs 885.T | CRL-7618 | abnormal |
| human | Hs 912.T | CRL-7661 | abnormal |
| human | Hs 938.T | CRL-7688 | abnormal |
| human | SW527 | CRL-7940 | Paget's disease |
| human | 184A1 | CRL-8798 | epithelium; |
| | | | chemically |
| | | | transformed |
| human | 184B5 | CRL-8799 | epithelium; |
| | | | chemically |
| | | | transformed |

TABLE V

| Species | Cell Line Name | ATCC No. | Description |
|---|---|---|---|
| human | RWPE-1 | CRL-11609 | transfected with Ki-MSV |
| human | RWPE-2 | CRL-11610 | transfected with HPV-18 |
| | | | and Ki-MSV |
| human | PWR-1E | CRL-11611 | immortalized with Ad12- |
| | | | SV40 hybrid virus |
| human | PZ-HPV-7 | CRL-2221 | epithelium; HPV-18 |
| | | | transformed |

TABLE VI

| Species | Cell Line Name | ATCC No. |
|---|---|---|
| human | Hs 1.Tes | CRL-7002 |
| human | Hs 181.Tes | CRL-7131 |

In one example of an appropriate neuroblastoma cell for use with the invention is human neuroblastoma cell SH-SY5Y as mentioned in the Examples.

The cells and cell lines disclosed herein can be transfected with one or more of the expression vectors described herein, for example, a sumoylation responsive promoter (e.g., tyrosine hydroxylase promoter) operably linked to a detectable reporter. Cells comprising such vectors can be used for the identification of compounds that modulate sumoylation. In brief, the method involves contacting a cell expressing a sumoylation-responsive promoter operably linked to a detectable reporter with a candidate compound; and detecting an alteration (e.g., increase or decrease) in the expression level of the detectable reporter. This alteration in reporter expression is identifies the compound as modulating sumoylation. In one embodiment, the compound modulates the sumoylation state of a polypeptide capable of acting as a sumoylation substrate and, dependent on its sumoylation status, binding to the sumoylation-responsive promoter (e.g., the polypeptide when sumoylated increases or decreases expression of the detectable reporter), such as the tyrosine hydroxylase promoter (GenBank Accession No. AF536811). Alternatively, the polypeptide when unsumoylated increases or decreases expression of the detectable reporter. As reported in more detail below, the tyrosine hydroxylase promoter is one exemplary sumoylation-responsive promoter that may be operably linked to a reporter construct. Expression of a reporter construct operably linked to the tyrosine hydroxylase reporter is repressed in the presence of sumoylated PSF. Expression constructs that feature the tyrosine hydroxylase promoter are known in the art and described by Kim et al Biochem Biophys Res Commun. 2003 Dec. 26; 312(4):950-7.

Typically, just one type of expression vector will be used to transfect the cells. The term “transfection” as used herein means an introduction of a foreign DNA or RNA into a cell by mechanical inoculation, electroporation, infection, particle bombardment, microinjection, or by other known methods. Alternatively, one or a combination of expression vectors can be used to transform the cells and cell lines. The term “transformation” as used herein means a stable incorporation of a foreign DNA or RNA into the cell, which results in a permanent, heritable alteration in the cell. A variety of suitable methods are known in the field and have been described. See e.g., Ausubel et al, supra; Sambrook, supra; and the Promega Technical Manual.

In particular invention embodiments, a cell or cell line of choice is manipulated so as to be stably transformed by an expression vector of the invention. In some invention embodiments, transient expression of the vector (e.g., for less than about a week, such as one or two days) will be more helpful. Cells and cell lines that are transiently transfected or stably transformed by one or more expression vectors disclosed herein will sometimes be referred to as “recombinant”. By the phrase “recombinant” is meant that the techniques used for making cell or cell line include those generally associated with making and using recombinant nucleic acids (e.g., electroporation, lipofection, use of restriction enzymes, ligases, etc.).

The invention also provides methods for detecting and in some cases analyzing compounds that increase the activity of one or more promoters (or functional portions thereof) bound by a regulatory protein, where the binding of that protein with the promoter is modulated by the sumoylation state of the protein. Certain of those compounds can be further selected if needed to identify those with therapeutic capacity to treat or prevent the above-described neurodegenerative conditions. Preferred detection and analysis methods include both in vitro and in vivo assays to determine the therapeutic capacity of agents to prevent, treat, prolong the onset of, or help alleviate the symptoms of such disorders.

### Test Compounds and Extracts

In general, compounds capable of modulating sumoylation are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., Jolui Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PhalmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckerman et al, J. Med. Chem. 37:2678, 1994; Cho et al., Science 261:1303, 1993; Carrell et al, Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:3 S6-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract of interest is identified, further fractionation of the positive lead extract is necessary to isolate chemical constituents responsible for the observed effect. Thus, the goal of the extraction, fractionation, and purification process is the careful characterization and identification of a chemical entity within the crude extract that alters the activity of a sumoylation substrate or a polypeptide involved in sumoylation. Methods of fractionation and purification of such heterogenous extracts are known in the art. If desired, compounds shown to be useful as therapeutics for the treatment of a neurodegenerative disease are chemically modified according to methods known in the art.

### Pharmaceutical Therapeutics

The invention provides a simple means for identifying compositions (including nucleic acids, peptides, small molecule inhibitors, and mimetics) capable of acting as therapeutics that modulate sumoylation for the treatment of a neurodegenerative disease. Accordingly, a chemical entity discovered to have medicinal value using the methods described herein is useful as a drug or as information for structural modification of existing compounds, e.g., by rational drug design. Such methods are useful for screening compounds having an effect on a neurodegenerative disease related to sumoylation.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of an neurodegenerative disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms of the neurodegenerative disease. Generally, amounts will be in the range of those used for other agents used in the treatment of a neurodegenerative disease, although in certain instances lower amounts will be needed because of the increased specificity of the compound. A compound is administered at a dosage that controls the clinical or physiological symptoms of a neurodegenerative disease as determined by a diagnostic method known to one skilled in the art, or using any that assay that measures the sumoylation state of a polypeptide associated with a neurodegenerative disease.

### Formulation of Pharmaceutical Compositions

The administration of a compound for the treatment of a neurodegenerative disease may be by any suitable means that results in a concentration of the therapeutic that, combined with other components, is effective in ameliorating, reducing, or stabilizing the neurodegenerative disease or a symptom thereof. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a neurodegenerative disease by using carriers or chemical derivatives to deliver the therapeutic agent to a particular cell type (e.g., neuronal cell at risk of cell death) whose function is perturbed in the neurodegenerative disease. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic (s), the composition may include suitable parenterally acceptable carriers and/or excipients. The active therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in the form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing an active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active neurodegenerative disease therapeutic substance). The coating may be applied on the solid dosage form in a similar manner as that described in Encyclopedia of Pharmaceutical Technology, supra.

At least two active neurodegenerative disease therapeutics may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may be constructed to release the active neurodegenerative disease therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosage

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about S, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Therapeutic Methods

The present invention provides methods of treating a neurodegenerative disease or symptoms thereof (e.g., cytotoxicity) by modulating the sumoylation of a polypeptide expressed in a neuronal cell at risk of cell death. The methods comprise administering a therapeutically effective amount of a pharmaceutical composition comprising a compound that modulates sumoylation identified using the methods described herein to a subject (e.g., a mammal such as a human). Thus, one embodiment is a method of treating a subject suffering from or susceptible to a neurodegenerative disease or symptom thereof. The method includes the step of administering to the subject a therapeutic amount of an amount of a compound herein sufficient to treat the disease or symptom thereof, under conditions such that the disease is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a neurodegenerative disease or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which sumoylation may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with sumoylation, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

### Example 1

### DJ-1 and PSF Transcriptionally Regulates the Human Tyrosine Hydroxylase Promoter

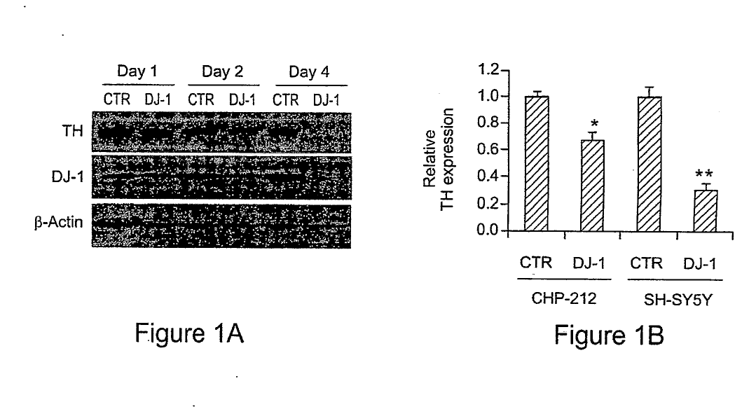

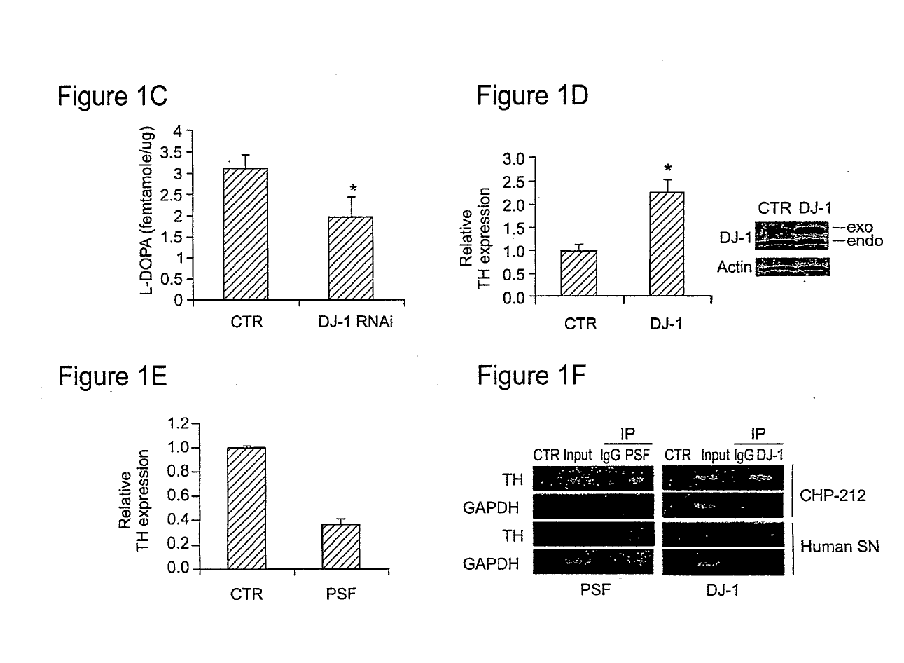

DJ-1 is a transcriptional co-activator. To determine whether DJ-1 regulated the expression of genes involved in dopaminergic neurotransmission, such as tyrosine hydroxylase, the rate-limiting enzyme that converts tyrosine to the dopamine precursor L-Dopa, DJ-1-specific siRNA constructs were used to inhibit the synthesis of endogenous DJ-1 in two human dopaminergic neuroblastoma cell lines, CHP-212 and SH-SY5Y cells. Expression of the DJ-1-specific siRNA mimicked the loss-of-function effects seen in Parkinson's disease patients with DJ-1 mutations. The protein levels of tyrosine hydroxylase and DJ-1 showed time-dependent decreases in CHP-212 cells transfected with DJ-1-specific siRNA (FIG. 1A). Four days after DJ-1 siRNA transfection, tyrosine hydroxylase protein expression was reduced by 90% (FIG. 1A). Quantitative real-time PCR results indicated that DJ-1 inactivation by siRNA significantly decreased the tyrosine hydroxylase mRNA levels in both CHP-212 and SH-SY5Y cells as determined by quantitative real-time PCR (FIG. 1B). In addition, the reduction in the tyrosine hydroxylase expression following siRNA knockdown of DJ-1 decreased the tyrosine hydroxylase activity by almost 40% in CHP-212 cells, as determined by the production of L-Dopa using HPLC (FIG. 1C). Consistent with these observations, the tyrosine hydroxylase mRNA expression was increased by more than 100% in SH-SY5Y cells stably expressing the human wild-type DJ-1 (FIG. 1D).

DJ-1 interacts with and blocks the functions of a transcriptional repressor PSF in human dopaminergic cells. As in SH-SY5Y cells, PSF specifically interacted with DJ-1 in untransfected CHP-212 cells. Therefore, to determine whether PSF repressed tyrosine hydroxylase transcription, wild-type PSF was transiently expressed in CHP-212 cells. The expression of wild-type PSF inhibited human tyrosine hydroxylase mRNA expression in CHP-212 cells (FIG. 1E). To confirm the transcriptional regulation of the human tyrosine hydroxylase promoter by both DJ-1 and PSF, chromatin immunoprecipitation (ChIP) assays were performed to assess the physical interactions between these two transcriptional regulators and the tyrosine hydroxylase promoter in vitro and in vivo. The DNA co-immunoprecipitated with either a monoclonal anti-PSF or a polyclonal anti-DJ-1 antibody using the lysates from CHP-212 cells or human substantia nigra pars compacta (SNpc) tissues were amplified by primers that specifically recognize the human tyrosine hydroxylase promoter, but not by primers recognizing the human GAPDH promoter (FIG. 1F). Taken together, these results strongly demonstrated that DJ-1 activates the human tyrosine hydroxylase expression and regulates dopamine synthesis.

### Example 2

### DJ-1 Inhibits the SUMOylation of PSF and its Repression of the Tyrosine Hydroxylase Promoter

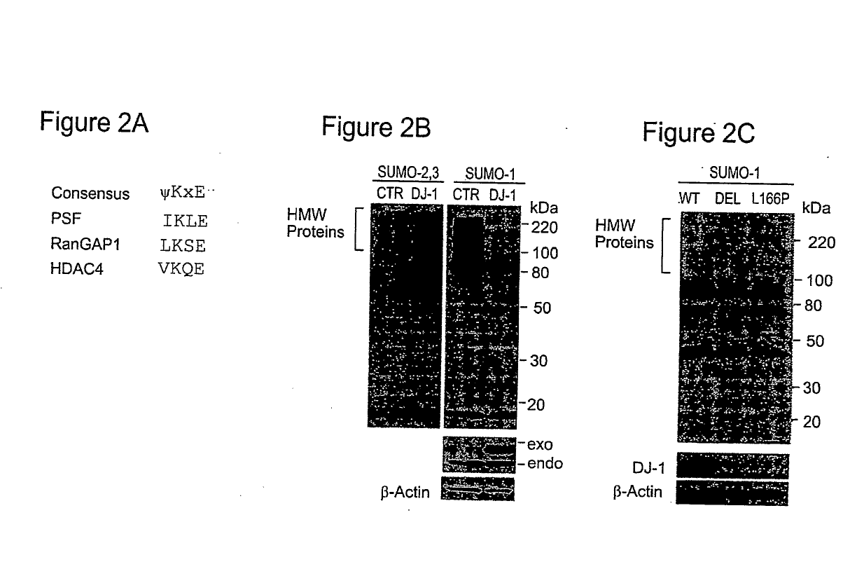

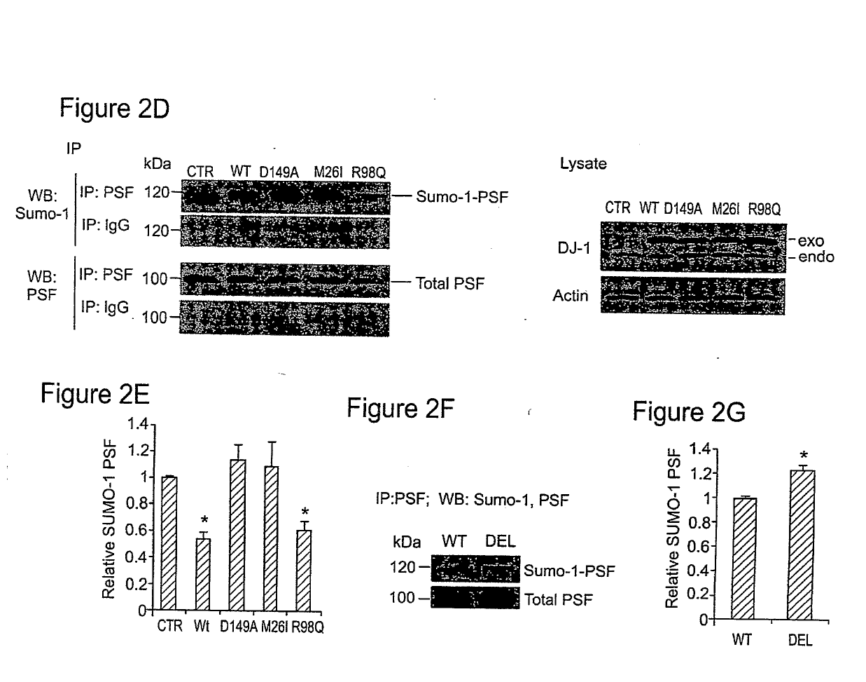

To elucidate the molecular mechanism of this transcriptional regulation, the possibility that DJ-1 might regulate the SUMOylation of PSF was assessed. DJ-1 interact with SUMO-1, SUMO activating enzyme Uba2 and conjugating enzyme ubc-9 using the yeast two-hybrid system. In addition, DJ-1 interacts with SUMO ligases PIASxa and PIASy, and potentially regulates their functions. PSF harbors a potential SUMOylation site (IKLE) that completely matches the consensus SUMOylation motif ψKxE, where the conserved lysine (K) and glutamic acid (E) are preceded by a hydrophobic amino acid (ψ_{.} and any amino acid (x), respectively (FIG. 2A). Recently, a proteomic study suggested that PSF is SUMOylated, although the site of modification has not been mapped (Rosas-Acosta et al., (2005) Mol Cell Proteomics 4, 56-72). First, the effect of DJ-1 on global SUMOylation in SH-SY5Y cells stably expressing the human wild-type DJ-1 was examined. Overexpression of the wild-type DJ-1 resulted in an overall decrease in the amount of high molecular weight proteins modified by SUMO-1, but not by SUMO-2 or SUMO-3 (FIG. 21B). To examine whether DJ-1 similarly regulates SuMOylation in vivo, the lymphoblast cells harvested from normal or PD patients carrying pathogenic mutations in the DJ-1 gene were analyzed. Consistent with the results from FIG. 2B, DJ-1 inactivation caused by the homozygous deletion of exons 1 to 5 (DEL) or the L166P point mutation resulted in a slight but reproducible increase in the amount of SUMO-1-modified high molecular weight proteins (FIG. 2C; fold change relative to WT, mean±SEM, DEL: 1.47±0.09; L166P: 3.07±0.61; p<0.05, n=3 for both conditions).

PSF was SUMOylation and DJ-1 's effect on this process was examined. Immunoprecipitation of PSF and western blotting of SUMO species and PSF using lysates from SH-SY5Y cells stably expressing various DJ-1 constructs indicated that PSF was modified by SUMO-1 (FIG. 2D), but not by SUMO-2 or SUMO-3. Furthermore, the wild-type and the non-pathogenic R98Q DJ-1, but not the pathogenic DJ-1 mutants expressed at similar levels, specifically reduced the abundance of SUMO-1-conjugated PSF (FIGS. 2D and 2E). The inability of the pathogenic DJ-1 mutants to suppress PSF SUMOylation suggested a functional link between the regulation of SUMOylation and Parkinson's disease pathogenesis. To examine the effect of DJ-1 inactivation on the SUMOylation of PSF in vivo, the abundance of SUMOylated PSF in the lymphoblast cells from a normal and a Parkinson's disease patient was evaluated using immunoprecipitation and western blotting. As shown in FIGS. 2F and 2G, the loss of DJ-1 resulted in minor but statistically significant accumulation of the SUMO-1-modified PSF. These results indicated that DJ-1 suppressed the modification of PSF by SUMO-1, and DJ-1-inactivating mutations may lead to the accumulation of SUMOylated proteins.

### Example 3

### Mutations at the PSF Sumoylation Consensus Abolish SUMO-1 Modification

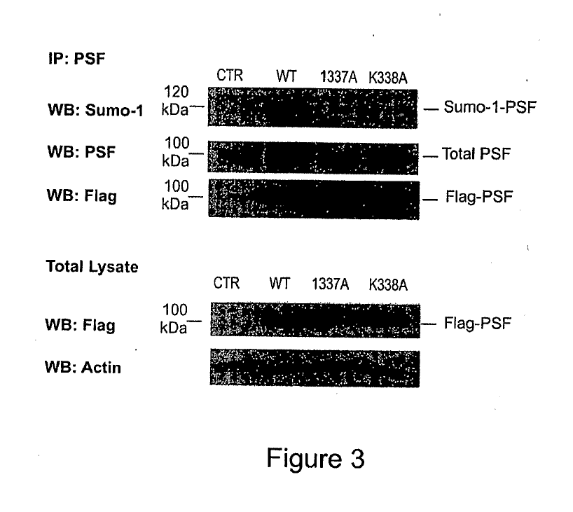

To determine whether the PSF sumoylation consensus sequence is required for PSF sumoylation, 1337A and K338A mutations were introduced within the PSF amino acid sequence. As shown in FIG. 3, mutations within the sumoylation consensus sequence of PSF abolished sumoylation.

### Example 4

### Mutations Decreased the Colocalization Between PSF and SUMO-1

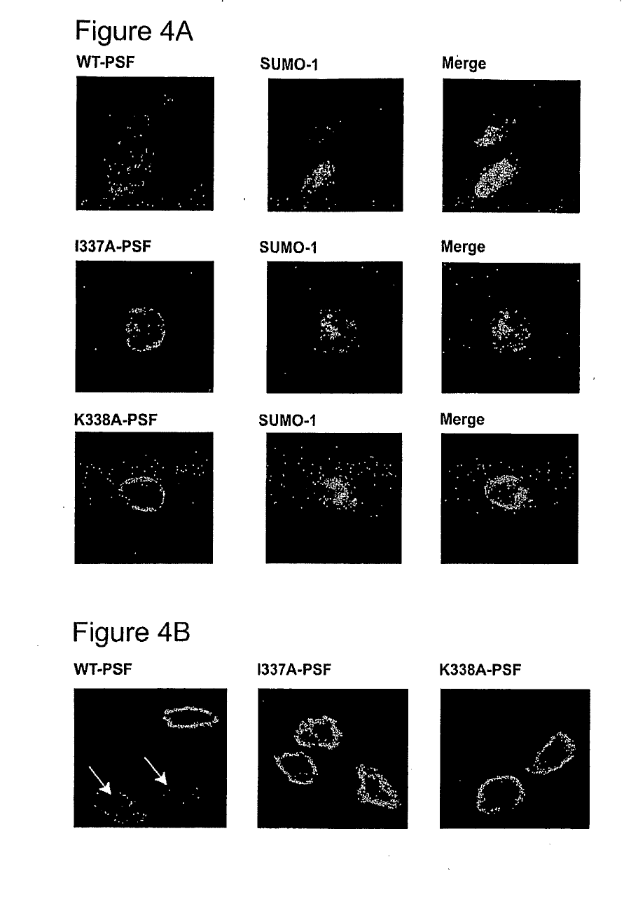

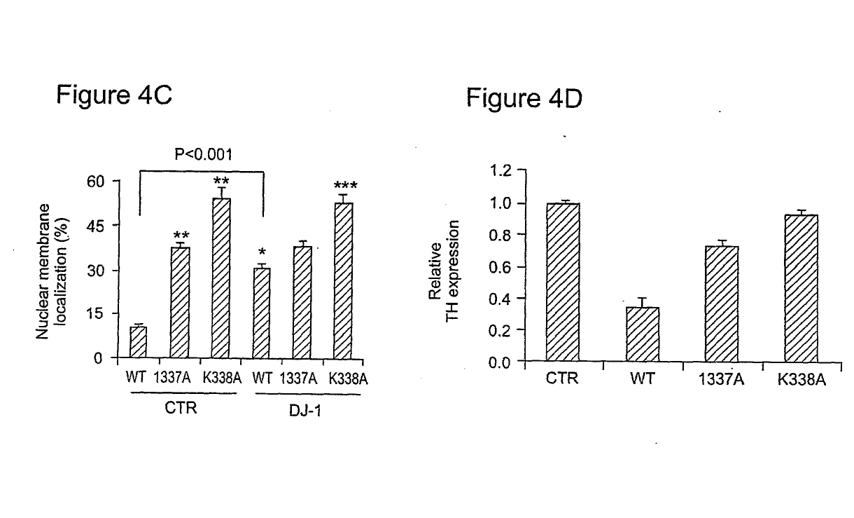

To characterize the sumoylation of PSF, the conserved lysine (K338A) and the preceding hydrophobic isoleucine (1337A) were mutated. Mutation at either site decreased the colocalization between PSF and SUMO-1 (FIG. 4A). Moreover, a significantly higher percentage of SUMO-deficient PSF mutants were localized proximal to the nuclear membrane instead of adopting a more diffused distribution in the nucleoplasma (FIGS. 4A, 4B and 4C). Quantification of the distribution patterns of the wild-type and mutant PSF in control cells or cells overexpressing DJ-1 indicated that DJ-1 promoted the translocation of wild-type PSF to the nuclear membrane, consistent with its ability to inhibit PSF sumoylation (FIG. 4C). In addition, DJ-1 failed to further increase the membrane bound SUMO-deficient PSF mutants (FIG. 4C), suggesting that DJ-1 regulates PSF sumoylation predominantly through the sumoylation of K338. To determine whether the sumoylation of PSF promoted its ability to repress tyrosine hydroxylase expression, mutations that abolish the consensus sumoylation site (K338A, 1337A) relieved the repression of tyrosine hydroxylase expression by PSF (FIG. 4D). Therefore, these observations indicated that DJ-1 promoted tyrosine hydroxylase transcription by preventing PSF sumoylation and altering PSF subnuclear localization.

While the molecular mechanism by which DJ-1 modulates sumoylation has not yet been defined, it appears that DJ-1 interacts with key components of the sumoylation machinery^{4,29}. Without wishing to be tied to one particular theory, it is possible that DJ-1 blocks access to SUMO substrates, such as PSF. Alternatively, DJ-1 may serve as a substrate to compete for sumoylation²⁹.

### Example 5

### DJ-1 Prevents the SUMOylation-Dependent Recruitment of HDAC1 by PSF

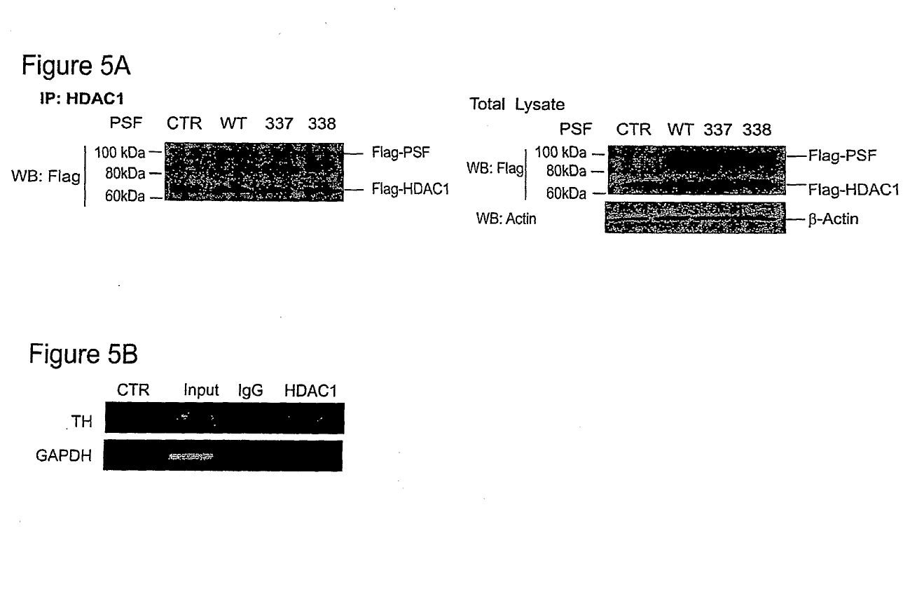

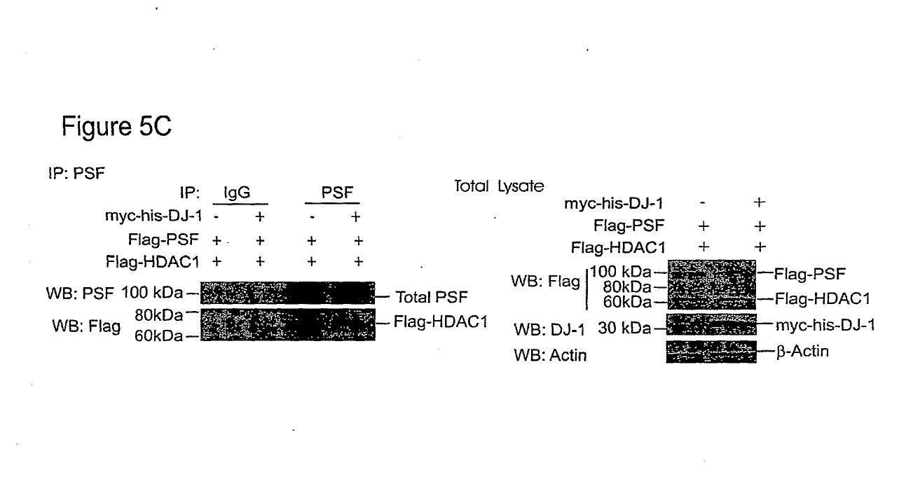

In most cases, SUMOylation of transcriptional regulators leads to increased transcriptional repression. One possible mechanism is the increased recruitment of histone deacetylase (HDAC) complexes to the promoter. HDAC complexes remove acetyl groups from histones and transcriptional factors, and repress transcription. In addition, PSF is known to repress nuclear hormone receptor-mediated transcription by recruiting Sin3A and the HDAC1. To examine the role of PSF SUMOylation in the recruitment of HDAC1, HEK293 cells were co-transfected with HDAC1 and the wild-type or SUMOylation-deficient PSF constructs, and then PSF was co-immunoprecipitated with an anti-HDAC1 antibody. Mutations that abolish the SUMOylation of PSF completely disrupted the interactions between PSF and HDAC1 (FIG. 5A). This result agrees entirely with the observation reported above showing that SUMOylation-deficient PSF mutants failed to repress the expression of tyrosine hydroxylase (FIG. 4D). To confirm the recruitment of HDAC1 to the human tyrosine hydroxylase promoter, HDAC1 and the wild-type PSF was co-expressed in HEK293 cells. ChIP assays were performed using an anti-HDAC1 antibody. Primers recognizing the human tyrosine hydroxylase promoter specifically amplified the DNA from the HDAC1 immuno-complex (FIG. 5B).

Since DJ-1 inhibited PSF SUMOylation, DJ-1 over-expression was assessed to determine whether this might prevent the recruitment of HDAC1 by PSF. Flag-tagged wt PSF and HDAC1 were co-transfected with an empty vector or the wild-type DJ-1 in Hela or HEK293 cells, and HDAC1 was co-immunoprecipitated with an anti-PSF antibody. Over-expression of DJ-1 disrupted the binding between PSF and HDAC1 in both Hela (FIG. 5C) and HEK293 cells. Taken together, this data suggested that by suppressing the SUMOylation of PSF, DJ-1 prevents the recruitment of the HDAC1 repressor complex to the human tyrosine hydroxylase promoter, and maintains an active transcription of tyrosine hydroxylase.

### Example 6

### HDAC Inhibitors Reversed the Inhibition of the Tyrosine Hydroxylase Promoter by DJ-1 Inactivation

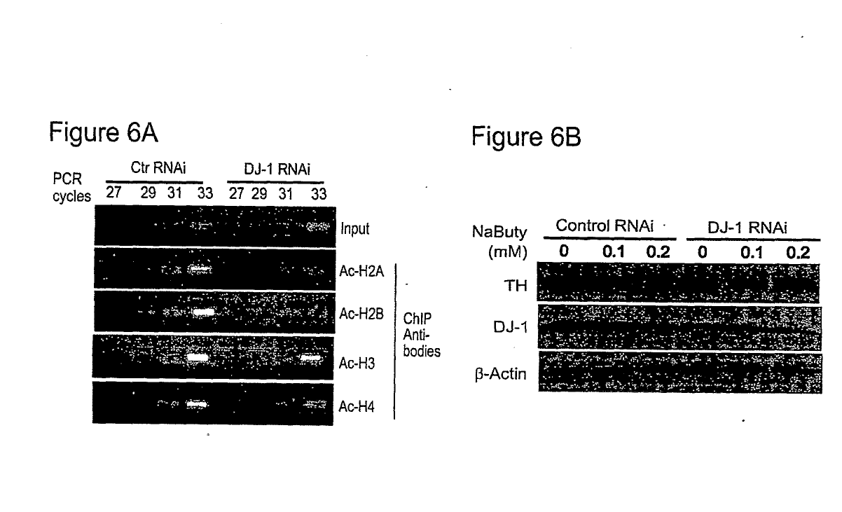

To validate the role of histone acetylation in DJ-1-mediated regulation of the human tyrosine hydroxylase promoter, CHP-212 cells were transfected with control or DJ-1-specific siRNA. ChIP assays were performed with antibodies that specifically recognize acetylated histones, and the human tyrosine hydroxylase promoter sequences were amplified using semi-quantitative PCR. Consistent with the concurrent inhibition of the tyrosine hydroxylase protein expression, DJ-1 inactivation caused a decrease in the amount of tyrosine hydroxylase promoter-associated acetylated histones, indicating transcriptional silencing (FIG. 6A). Since DJ-1 acted as a transcriptional co-activator eventually promoting histone acetylation, it was likely that chemical inhibitors of HDAC would reverse the effects of DJ-1 inactivation. Therefore, CHP-212 cells were pre-transfected with control or DJ-1-specific siRNA, and the cells were treated with increasing amounts of the HDAC inhibitor sodium butyrate. Sodium butyrate restored tyrosine hydroxylase expression (to 88% of the levels in the control at 0.2 mM) even in the presence of DJ-1 siRNA (FIG. 6B). In separate experiments, another HDAC inhibitor trichostatin A similarly reversed the tyrosine hydroxylase inhibition caused by DJ-1 inactivation.

### Example 7

### Wild-Type DJ-1 Blocked PSF-Mediated Gene Silencing and Apoptosis

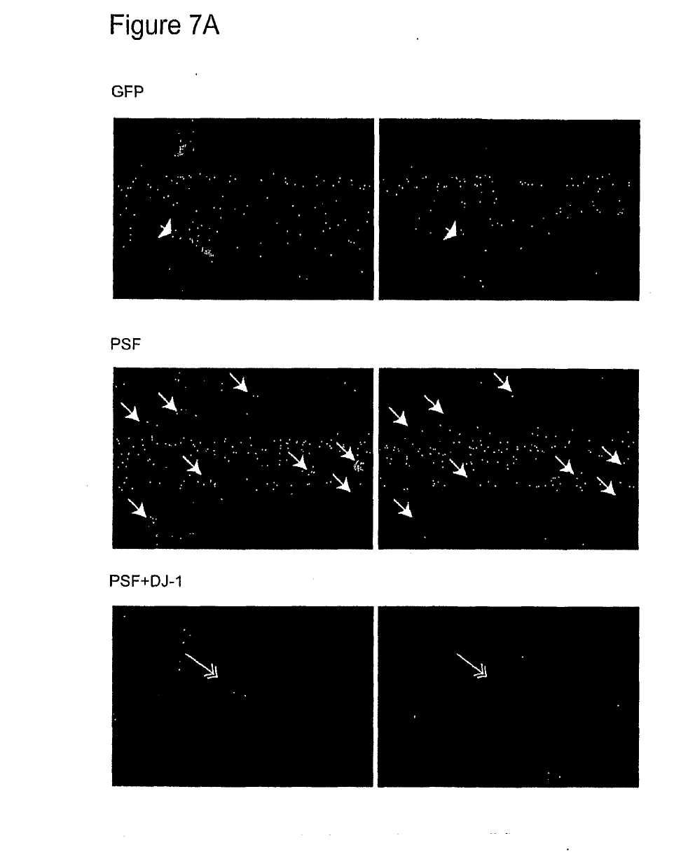

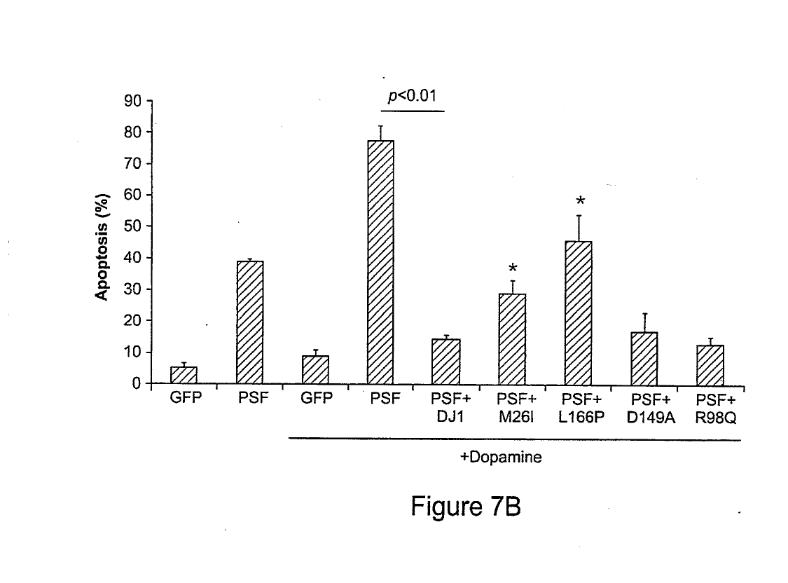

Loss-of-function mutations of DJ-1 associated with Parkinson's disease may increase transcriptional repression by PSF. To determine whether this could predispose cells to neurodegeneration, SH-SY5Y cells were transfected with a PSF expression vector, and then exposed to dopamine, a source of oxidative stress implicated in Parkinson's disease. Expression of PSF, but not a control green fluorescent protein (GFP) vector, markedly increased neuronal apoptosis (FIG. 7A, 7B: graph bars 1 and 2). After treatment with dopamine, more than 70% of PSF expressing cells were apoptotic (FIGS. 7A and 7B). Co-expression of the wild type DJ-1 or the non-pathogenic R98Q DJ-1 mutant abolished PSF-induced apoptosis (FIG. 7B, graph bars 5 and 9). Parkinson's disease -associated DJ-1 mutants were less effective in blocking PSF-mediated toxicity (FIG. 7B, columns 6-8), consistent with their reduced transcriptional activity. These data indicated that PSF not only repressed gene expression, but also induced neuronal apoptosis and sensitized SH-SY5Y cells to dopamine-induced cell death. Wild-type and the non-pathogenic R98Q DJ-1, but not the pathogenic DJ-1 mutants, effectively blocked PSF-mediated gene silencing and apoptosis.

### Example 8

### Wild Type DJ-1 Inhibited α-Synuclein-Induced Apoptosis

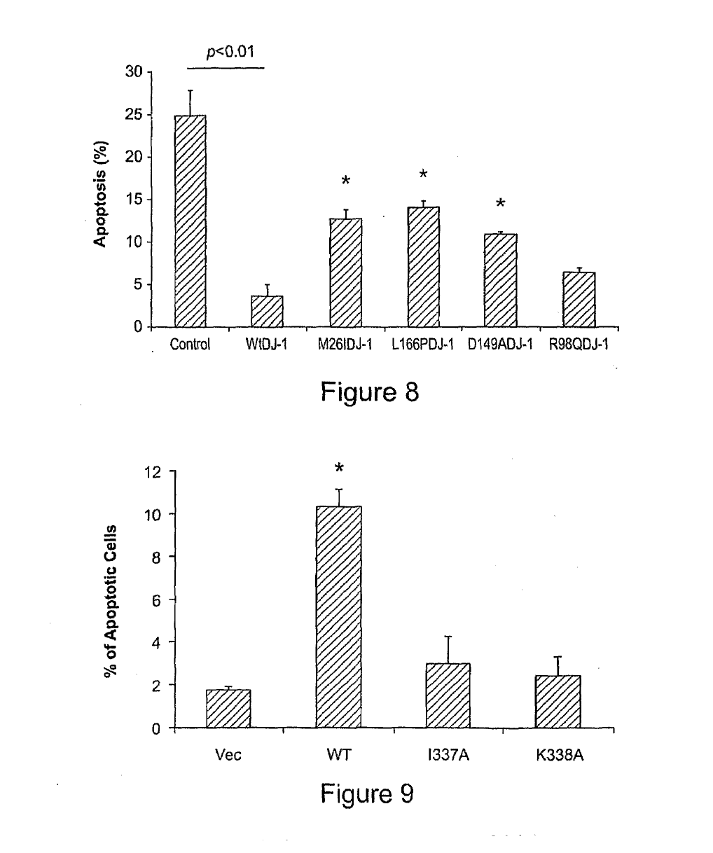

Accumulation of α-synuclein causes dopaminergic neuronal cell death and directly leads to Parkinson's disease, likely due to increased oxidative stress signals. To examine whether DJ-1 is neuroprotective against α-synuclein, dopaminergic SH-SY5Y cells were co-transfected with either GFP (control), wild type human DJ-1 or the indicated DJ-1 mutants together with A30P α-synuclein (FIG. 8). Over-expression of mutant A30P α-synuclein in native SH-SY5Y cells induced neuronal cell death (FIG. 8, column 1). Wild type and R98Q DJ-1 significantly inhibited A30P α-synuclein-induced apoptosis (FIG. 8, columns 2 and 6). Three pathologic DJ -1 mutants, including M261, L166P, and D149A, showed reduced neuroprotective activity against α-synuclein.

### Example 9

### SUMO-Deficient PSF do not Induce Apoptosis

Wild-type PSF induced apoptosis in transiently transfected SH-SY5Y cells (FIG. 9). Interestingly, PSF having a mutation in the sumoylation consensus sequence did not induce apoptosis in transiently transfected SH-SY5Y cells (FIG. 9). These results indicate that sumoylation of PSF is required for the induction of apoptosis.

Taken together, these experiments demonstrated that DJ-1 transcriptionally up-regulates human tyrosine hydroxylase expression and prevent apoptosis by modulating sumoylation, thus providing a mechanism for the selective vulnerability observed in the dopaminergic pathway as a result of loss of DJ-1 functions. Additionally, these findings highlight the role of transcriptional regulation in the pathogenesis of neurodegenerative diseases, and suggest disease-modifying therapeutic approaches by targeting sumoylation.

The experiments described above were carried out as follows.

### Cell Culture, Plasmids And Chemicals.

Human CHP-212 cells were purchased from ATCC and maintained in cell culture media, EMEM/F-12 (50%/50%) containing 10% FBS and antibiotics. Native SH-SY5Y cells were maintained in DMEM supplemented with 10% FBS and antibiotics. For immunofluorescence, cells were grown on coverslips. Wild-type and mutant DJ-1 constructs and SH-SY5Y cells stably expressing these constructs were described previously⁸. Rat tyrosine hydroxylase-luc reporter plasmid (Kim et al Biochem Biophys Res Commun. 2003 Dec. 26; 312(4):950-7) and the pTK-Renilla luciferase plasmid for transfection control was obtained from Promega (Madison, Wis.). A Flag tag was fused inframe to the N-terminus of human wild-type PSF⁸ to generate tagged PSF construct (pCMV-Flag-PSF). To introduce point mutations at the PSF sumoylation site (1337A and K338A), a two-step PCR mutagenesis approach was used to mutate and amplify the PSF fragments flanked by the EcoRI and BamHI sites. The resulting PCR products (979 bp) were then subcloned into an expression vector using pCMV-Flag-PSF as backbone. The PSF plasmids were confirmed by sequencing. The sequence of the flanking primers: forward: 5′-gatgtcggttgtttgttg-3′; reverse: 5′-atctcccatgttcattgct-3′. Mutagenesis primers for PSF-1337A: forward: 5′-ggattcggatttgctaagcttgaatctagagc-3′; reverse: 5′-gctctagattcaagcttagcaaatccgaatcc-3′. Mutagenesis primers for PSF-K338A: forward: 5′-ggattcggattattgcgcttgaatctagagc-3′; reverse: 5′-gctctagattcaagcgcaataaatccgaatcc-3′. Trichostatin A (TSA) was from Sigma (St. Louis, Mo.).

Transfection of siRNA and Plasmids.

CHP-212 cells or SH-SY5Y cells were plated in six-well culture dishes and transfected with 100 nM of siRNA against human DJ-1 constructs (SMARTpool reagent, Dharmacon, Lafayette, Colo.) or non-specific control siRNA constructs (siControl non-targeting pool, Dharmacon). The siRNA was transfected in to cells using the cationic lipid Transfectin reagent (Bio-Rad, Hercules, Calif.) following the manufacturer's suggested protocol. Cells were harvested forty-eight hours post-transfection for RNA extraction or at Day 1, 2, or 4 for Western blot. To analyze the effects of trichostatin (TSA) on the tyrosine hydroxylase expression after DJ-1 inactivation, the indicated amount of TSA was added to fresh medium at forty-eight hours post-transfection and cultured for an additional two days with one change of fresh medium containing TSA. Lipofectamine 2000 (Invitrogen, Carlsbad, Calif.) was used to transfect various plasmids.

To study the transcriptional repression of tyrosine hydroxylase by PSF, CHP-212 cells plated in 10 cm dishes were co-transfected with 20 μg of control vector, wild-type or mutant PSF plasmids, and 2 μg of GFP. Cells were harvested at 48 hours after transfection, and the transfected cells were enriched using a Cytomation Mo-Flo cell sorter (Core facility, Dana Farber Cancer Institute, Boston) for subsequent total RNA extraction and mRNA analysis using Q-PCR.

### Western Blotting, Immunoprecipitation and Antibodies.

The procedures for western blotting and immunoprecipitation were described previously¹⁹. For SUMO-1-conjugated PSF and DJ-1 immunoprecipitation, cells were lysed in denaturing RIPA-DOC buffer (50 mM Tris-HCl buffer (pH 8.0), containing 150 mM NaCl, 1% Triton X-100, 0.1% SDS, 1% sodium deoxycholate, 10 mm EDTA and a protease inhibitor cocktail (1× protease cocktail, Roche (Indianapolis, Ind.). For endogenous DJ-1 and PSF co-immunoprecipitation, cells were lysed in non-denaturing lysis buffer containing 1% Triton-X100. Antibodies used for immunoprecipitation included a mouse monoclonal anti-PSF (Sigma Chemical, St. Louis, Mo.) and a rabbit polyclonal anti-DJ-18. Antibodies for western blotting included a mouse monoclonal anti-tyrosine hydroxylase (Sigma Chemical, St. Louis, Mo.); monoclonal (Stressgen, San Diego, Calif.) and polyclonal anti-DJ-1; a goat anti-β-actin (Santa Cruz Biotechnology, Santa Cruz, Calif.); a mouse monoclonal anti-SUMO-1, a rabbit polyclonal antibody that recognizes anti-SUMO-2/3 (Invitrogen, San Diego, Calif.); and a rabbit polyclonal antibody that recognizes anti-acetylated histones (Histone sampler kit, Cell signaling, Beverly, Mass.);

### RNA Extraction and Real-Time Quantitative PCR (Q-PCR).

RNA was extracted using a mono-phasic solution of phenol and guanidine isothiocyanate that is commercially available as Trizol reagent (Invitrogen) and purified with a commercially available silica-gel-based membrane, the RNeasy Kit or RNeasy Micro Kit (Qiagen, Germany), and quantified with a spectrophotometer. The quality of RNA was confirmed by agarose gel electrophoresis. The reference RNA used for calibration curve was made by pooling equal amount of RNA from all samples. Q-PCRs were performed using a LightCycler (Roche, Indianapolis, Ind.) and One-Step QuantiTect™ SYBR Green RT-PCR kit (Qiagen) that provides for kinetic quantification of PCR products. Kinetic quantification of real-time PCR allows the course of a polymerase chair reaction to be visualized as a curve that contains an initial lag phase, an exponential (log-linear) phase, and a final plateau phase. Experimental conditions and primer design parameters were set in accordance with the manufacturer's instructions. Primers for Q-PCR were designed to have an amplicon size of 100-200 bps. Agarose gel electrophoresis was used to confirm the specificity of PCR reactions. Results were normalized to an internal control PCR amplified with GAPDH or β-Actin primers included in the same run of Q-PCR. Primers for the human tyrosine hydroxylase: Forward: 5′-cctcgcccatgcactc-3′; Reverse: 5′-cctcgcccatgcactc-3′. Primers for PSF: Forward: 5′-accaccagcagcatcacc-3′; Reverse: 5′-tcccaacaaacaaccgaca-3′.

### Chromatin Immunoprecipitation (ChIP) Assays.

Chromatin Immunoprecipitation (CHIP) assays were performed using a commercially available kit, the EZ CHIP Kit (Upstate, Charlottesville, Va.), that includes lysis buffer to lyse formaldehyde-treated cells prior to sonication, a protein A agarose slurry that precipitates antibody-protein-DNA complexes, several wash buffers that are necessary for reducing non-specific background interactions, and a 5M NaCl solution that reverses the formaldehyde cross-links in accordance with the manufacturer's instructions with the following modifications. After protein-DNA cross-linking and harvesting, the cell pellets were resuspended in lysis buffer and sonicated on ice using a Branson Digital Sonifier (Branson Ultrasonics Corporation, CT) with 16 sets of 4-second pulses at 17% of maximum power. The genomic DNA was sheared to 300-1200 bp in length. Aliquots of chromatin solution (each equivalent to 1×10⁶ cells) were precleared with Protein G agarose and incubated with species-matched IgG or specific antibodies overnight at 4° C. with rotation. The antibodies used in the CHIP assays for DJ-1, PSF and acetylated histones were described above. The final immunoprecipitated DNA fragments were used as templates for PCR with a commercially available recombinant Taq DNA polymerase complexed with a proprietary antibody that blocks polymerase activity at ambient temperatures, hot start Platinum Taq, (Invitrogen, San Diego, Calif.) using the following conditions: 3 minutes at 94° C.; 32 cycles of 30 seconds denaturation at 95° C., 30 seconds annealing at 57° C. and 30 seconds elongation at 72° C.; with one final incubation for 2 minutes at 72° C. For semi-quantitative PCR, 27, 29, 31 and 33 cycles were used. The Primer 3 software was used to design the PCR primers for amplifying the human tyrosine hydroxylase promoter. The primers for ChIP using anti-DJ-1, PSF, and acetyl-histones: Forward: 5′-gagccttcctggtgtttgtg-3′, and reverse: 5′-ctctccgattccagatggtg-3′. The primers for ChIP using anti-AR: Forward: 5′-gggtcttccctttgctttga-3′, and reverse: 5′-cctgggacctttcctaaaactg-3′. The PCR products were analyzed by electrophoresis on commercially available 2% TAE agarose gels.

### Immunofluorescence and Confocal Microscopy.

Cells grown on coverslips were fixed with 4% parafomaldehyde and incubated with a rabbit polyclonal anti-Flag (1:200, Sigma) and/or a mouse monoclonal anti-HA (1:200, Santa Cruz), followed by incubation with Alexafluor 488 or 594-conjugated secondary antibodies (1:300, Invitrogen). Images were captured with a Nikon DiaPhot fluorescent or a Zeiss Axiophot confocal microscope.

### Transfection, Immunofluorescence Microscopy and Analysis of Neuronal Apoptosis

SH-SY5Y cells plated on coverslips in 24 well dishes were transfected using Lipofectamine 2000 (Invitrogen) or Transfectin (Bio-Rad) reagents. To assess the protective effect of DJ-1 on α-synuclein toxicity, 2 μg of A30P α-synuclein was co-transfected with 1 μg of wild type or mutant DJ-1 expression plasmids. Similarly, 1 μg of PSF was co-transfected with 1 μg of DJ-1 plasmids in PSF toxicity assays.

Cell Transfection with K338A and 1337A Mutant PSF

Native SH-SY5Y cells plated in 10 cm dishes were co-transfected with 20 μgs of pcDNA3 (CTR), Flag-tagged wild-type human PSF (WT), I337A-PSF(I337A) or PSF-K338A(I338A), and 10 μgs of SUMO-1 plasmid and 10 μgs of PIASy plasmid. 48 hours after transfection, cells were lysed in RIPA buffer containing protease inhibitors.

### Apoptosis Quantitation

Quantitative analysis of apoptosis was induced by wild-type or mutant PSF as described above. SH-SY5Y cells transfected with 0.5 μg of control or indicated Flag-tagged PSF constructs were fixed and double-labeled with anti-Flag, and the Hoechst dye forty-eight hours post-transfection for apoptosis analysis as described by Xu et al., Human Mol Genetics 2005. n=3, with more than 100 cells scored per condition. *: P<0.01 relative to control.

### Statistical Analysis.

Statistical analyses were performed using InStat 3.0 (GraphPad, San Diego, Calif.). P values, sample numbers and statistical tests used were indicated in the figure legends.

A review of the following specific references will help advance appreciation of the present invention.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Bonifati, et al., (2003) Science 299:256-9
2. Taira, et al., (2004) EMBO Rep 5:213-218
3. Yokota, et al., (2003) Biochem Biophys Res Commun 312:1342-8
4. Martinat, et al., (2004) PLoS Biol 2, e327
5. Shendelman, et al., (2004) PLoS Biol 2, e362
6. Xu, et al. (2005) Hum Mol Genet 14:1231-1241
7. Junn, et al., (2005) Proc Natl Acad Sci USA 102:9691-6
8. Canet-Aviles, et al., (2004) Proc Natl Acad Sci USA 101:9103-8
9. Niki, et al., (2003) Mol Cancer Res 1:247-61
10. Takahashi, et al., (2001) J Biol Chem 276:37556-63
11. Johnson, et al., (2004) Annu Rev Biochem 73:355-82
12. Melchior, et al., (2003) Trends Biochem Sci 28:612-8
13. Gill, G. (2004) Genes Dev 18:2046-59
14. Girdwood, et al., (2004) Semin Cell Dev Biol 15:201-10
15. Shinbo, et al., (2005) Cell Death Differ
16. Ross, C.A. (2002) Neuron 35:819-22
17. Okazawa, H. (2003) Cell Mol Life Sci 60:1427-39
18. Steffan, et al., (2004) Science 304:100-4
19. Schmidt, et al., (2003) Cell Mol Life Sci 60:2561-74
20. Shav-Tal, et al., (2002) FEBS Lett 531:109-14
21. Patton, et al., (1993) Genes Dev 7:393-406
22. Gozani, et al., (1994) Embo J 13:3356-67
23. Ishitani, K. et al., (2003) Biochem Biophys Res Commun 306:660-5
24. Mathur, et al., (2001) Mol Cell Biol 21:2298-311
25. Yang, et al., (2004) Mol Cell 13:611-7
26. Peterson, et al., (2004) Curr Biol 14:R546-51
27. Wang, et al., (2004) Novartis Found Symp 259:238-45; discussion 245-8-285.8
28. Hely, et al., (2000) Neurosci 7:484-94
29. Lang, et al., (1998) N Engl J Med 339:1044-53
30. Dunnett, et al., (1999) Nature 399:A32-9

1. A method for identifying a compound useful for the treatment of a neurodegenerative disease, the method comprising:
(a) contacting a cell with a candidate compound; and
(b) identifying a decrease in sumoylation of a sumoylation substrate in the cell, wherein a compound that decreases sumoylation relative to a reference is a compound that treats a neurodegenerative disease.

2. The method of claim 1, wherein the sumoylation substrate is selected from the group consisting of PSF, huntingtin, androgen receptor and amyloid precursor protein.

3. The method of claim 1, wherein the neurodegenerative disease is selected from the group consisting of Parkinson's disease, Huntington's Disease, Alzheimer's disease, Kennedy's Disease, and spinocerebellar ataxia.

4-11. (canceled)

12. A method for identifying a compound that increases tyrosine hydroxylase expression, the method comprising:
(a) contacting a cell with a compound; and
(b) identifying a decrease in a sumoylated polypeptide in the cell, wherein a compound that decreases the amount of sumoylated polypeptide relative to a reference is a compound that increases tyrosine hydroxylase expression.

13. A method for reducing apoptosis, the method comprising:
(a) contacting a cell at risk of apoptosis with a compound; and
(b) identifying a decrease in a sumoylated polypeptide in the cell, wherein a compound that decreases the amount of sumoylated polypeptide relative to a reference is a compound that reduces apoptosis.

14. A method for identifying a compound useful for the treatment of a neurodegenerative disease, the method comprising:
(a) contacting a PSF-expressing cell with a compound; and
(b) identifying a decrease in sumoylated PSF in the cell, wherein a compound that decreases the amount of sumoylated PSF relative to a reference is a compound that treats a neurodegenerative disease.

15-20. (canceled)

21. A method for identifying a compound that increases tyrosine hydroxylase expression or decreases apoptosis, the method comprising:
(a) contacting at least a fragment of a PSF polypeptide comprising a sumoylation consensus sequence with a compound under conditions that permit binding; and
(b) detecting binding of the compound to the consensus sequence, wherein a compound that specifically binds the PSF polypeptide increases tyrosine hydroxylase expression or decreases apoptosis.

22. The method of claim 21, wherein the PSF polypeptide comprises at least the following amino acid sequence: ψKXD, wherein ψ is a hydrophobic amino acid, K is lysine, X is any amino acid, and E is a glutamic acid.

23-31. (canceled)

32. A method of identifying a compound that modulates sumoylation, the method comprising:
(a) contacting a cell expressing a sumoylation-responsive promoter operably linked to a detectable reporter with a candidate compound; and
(b) detecting an alteration in the expression level of the detectable reporter, thereby identifying the compound as modulating sumoylation.

33-39. (canceled)

40. A method of identifying a compound that treats Parkinson's disease, the method comprising:
(a) contacting a cell expressing a tyrosine hydroxylase promoter operably linked to a detectable reporter with a candidate compound; and
(b) detecting an alteration in the expression level of the detectable reporter, thereby identifying the compound as modulating sumoylation.

41. A method for identifying a compound that reduces PSF sumoylation, the method comprising:
(a) contacting a PSF polypeptide comprising a sumoylation consensus sequence with a compound under conditions that permit SUMO ligation; and
(b) detecting a decrease in PSF sumoylation in the presence of the compound relative to a control condition.

42-43. (canceled)

44. A method for identifying a compound that increases cleavage of SUMO from a sumoylated polypeptide, the method comprising:
(a) contacting at least a fragment of a sumoylated polypeptide with a compound under conditions that permit cleavage of a SUMO moiety;
(b) detecting a decrease in the level of sumoylation in the presence of the compound relative to a reference.

45-49. (canceled)

50. A method for preventing or ameliorating a neurodegenerative disease selected from the group consisting of Parkinson's disease, Huntington's Disease, Kennedy's Disease, and spinocerebellar ataxia in a subject in need thereof, the method comprising administering to the subject a compound that decreases sumoylation.

51-52. (canceled)

53. A method for preventing or ameliorating Parkinson's Disease in a subject, the method comprising administering to the subject an effective amount of a histone deacetylase inhibitor that increases tyrosine hydroxylase expression.

54. The method of claim 53, wherein the tyrosine hydroxylase inhibitor is selected from the group consisting of trichostatin A, sodium butyrate, and suberoylanilide hydroxamic acid (SAHA).

55. A method for preventing or ameliorating Parkinson's Disease in a subject, the method comprising administering to the subject an effective amount of a compound that decreases PSF sumoylation.

56-63. (canceled)

64. A kit for the treatment or prevention of a neurodegenerative disease, the kit comprising a compound that decreases sumoylation.

65. A kit for the treatment or prevention of a neurodegenerative disease, the kit comprising a compound that increases tyrosine hydroxylase expression.

66. A pharmaceutical composition comprising a compound that increases tyrosine hydroxylase selected from the group consisting of sodium butyrate, trichostatin A, and SAHA.

67. A packaged pharmaceutical comprising:
(a) an effective amount of an agent that reduces sumoylation of a sumoylation substrate; and
(b) instructions for using the agent to treat a neurodegenerative disease.
